(19) United States
(12) Patent Application Publication
(10) Pub. No.: US 2024/0110160 A1
(43) Pub. Date: Apr. 4, 2024

(54) A TRANS-COMPLEMENTATION SYSTEM FOR SARS-COV-2

(71) Applicant: Board of Regents, The University of Texas System, Austin, TX (US)

(72) Inventors: PEI-YONG SHI, Galveston, TX (US); XUPING XIE, Galveston, TX (US); XIANWEN ZHANG, Galveston, TX (US)

(21) Appl. No.: 18/273,266

(22) PCT Filed: Jan. 14, 2022
(86) PCT No.: PCT/US2022/012604

§ 371 Date: Jul. 19, 2023

Related U.S. Application Data
(60) Provisional application No. 63/138,063, filed on Jan. 15, 2021.

(51) Int. Cl. C12N 7/00; C07K 14/005; C12N 15/86

(52) U.S. Cl. CPC C12N 7/00; C07K 14/005; C12N 15/86; C12N 2740/15043; C12N 2770/20022; C12N 2770/20051; C12N 2830/002

(57) ABSTRACT

Certain embodiments are directed to a trans-complementation system, system components, and method of using the same for SARS-CoV-2 that can be performed at BSL-2 laboratories for COVID-19 research and countermeasure development. The system thus can be used by researchers in industry, academia, and government laboratories who lack access to BSL-3 facility. This approach also can be applied to other coronaviruses.

### PRIORITY PARAGRAPH

This application claims priority to U.S. Provisional Application Ser. No. 63/138,063 filed Jan. 15, 2021, which is incorporated herein by reference in its entirety.

### STATEMENT REGARDING FEDERALLY FUNDED RESEARCH

This invention was made with government support under AI142759, AI134907, AI145617, and TR001439 awarded by the National Institutes of Health (NIH). The government has certain rights in the invention.

### BACKGROUND

Three zoonotic betacoronaviruses have emerged as causing global epidemics and pandemics in less than twenty years: severe acute respiratory syndrome coronavirus (SARS-CoV) in 2002, Middle East respiratory syndrome coronavirus (MERS-CoV) in 2012, and SARS-CoV-2 in 2019 (1). The coronavirus disease 2019 (COVID-19) pandemic has caused unprecedented social and economic disruption. As of Jan. 13, 2021, SARS-CoV-2 has infected over 93 million people, leading to almost 2 million deaths (see URL worldometers.info/coronavirus/). In response to the pandemic, the scientific community has rapidly developed experimental platforms to study COVID-19 and develop countermeasures. Several groups have established infectious cDNA clones and reporter SARS-CoV-2 to facilitate the development and analysis of first-generation vaccines and therapeutics (2-6). However, since SARS-CoV-2 is a biosafety level-3 (BSL-3) pathogen, the requirement of high containment represents a bottleneck for antiviral and vaccine evaluation. Thus, a BSL-2 cell culture system that recapitulates authentic viral replication is urgently needed.

The genome of SARS-CoV-2 is a positive-sense, single-stranded RNA of 30 kb in length. SARS-CoV-2 virion consists of an internal nucleocapsid [formed by the genomic RNA coated with nucleocapsid (N) proteins] and an external envelope [formed by a bilipid membrane embedded with spike (S), membrane (M), and envelope (E) proteins] (7). The genomic RNA encodes open-reading-frames (ORFs) for replicase (ORF1a/ORF1b), S, E, M, and N proteins, as well as seven additional ORFs for accessory proteins (1). Stable cell lines containing self-replicative replicons have been developed for many viruses, including coronaviruses (8-12). Because replicons lack structural genes, they are not infectious and can safely be manipulated in BSL-2 laboratories. For SARS-CoV-2, although a transient replicon system has been established (13); however, no stable replicon cell line has been reported.

### SUMMARY

A solution to the problems outlined above includes the development a single-round infectious SARS-CoV-2 through trans-complementation (i.e., a replication defective SARS-CoV-2 virus). The single-round SARS-CoV-2 described herein is engineered with a reporter gene (and/or other heterologous nucleic acid segment) that facilitates high-throughput antiviral screening and neutralizing antibody measurement. The safety of the system in cell culture, hamsters, and highly susceptible human angiotensin-converting enzyme 2 (hACE2) transgenic mice was assessed. Results suggest that the trans-complementation system can be used safely at BSL-2 laboratories.

The requirement for biosafety level-3 (BSL-3) containment to culture severe acute respiratory syndrome coronavirus 2 (SARS-CoV-2) poses a problems for research and countermeasure development. Herein described is a trans-complementation system that produces single-round infectious SARS-CoV-2 that recapitulates authentic viral infection and replication. The single-round infectious SARS-CoV-2 described can be used for high-throughput neutralization and antiviral testing at BSL-2 laboratories. The trans-complementation system consists of two components: (i) a genomic viral RNA containing a deletion of ORF3 and envelope genes (ΔORFe/E) and (ii) a producer cell line expressing the two deleted genes (ORF3 and E). Trans-complementation of the two components generates virions that can infect naive cells for one round or at a level that does not result in disease; but does not produce wild-type or mutant SARS-CoV-2 capable of efficiently infecting normal cells for multiple rounds or at levels that cause disease. Hamsters and K18-hACE2 transgenic mice inoculated with the complementation-derived virions (virions with a ΔORFe/E SARS-CoV-2 genome) exhibited no signs of disease, even after the mice were inoculated intracranially with the highest possible dose. In contrast, animals inoculated with wild-type SARS-CoV-2 developed significant disease and/or death. These results suggest that the trans-complementation platform can be safely used at BSL-2 laboratories, allowing the system to be used widely for research and countermeasure development, particularly for investigators who lack access to BSL-3 facility.

Certain embodiments described herein are directed to viral genomes, producer cell lines, systems, and methods for producing and using ΔORFe/E SARS-CoV-2 genomes, virions, and producer cells.

Certain embodiments are directed to trans-complementation system comprising: (i) a ΔORF3/E SARS-CoV-2 genomic viral RNA having ORF3 and envelope genes deleted; and (ii) a stable producer cell line expressing the SARS-CoV-2 ORF3 and envelope genes, wherein the producer cell line expresses a SARS-CoV-2 ORF3 gene and a SARS-CoV-2 Envelope gene. The ΔORF3/Envelope SARS-CoV-2 genomic viral RNA can further comprise a heterologous nucleic acid segment encoding a reporter gene. The SARS-CoV-2 ORF3 gene and a SARS-CoV-2 Envelope gene can be under the control of an inducible promoter, i.e., expression of these genes is inducible.

Certain embodiments are directed to a replication defective SARS-CoV-2 RNA genome comprising a deletion of the ORF3 and envelope genes (ΔORF3/E SARS-CoV-2), See SEQ ID NO:2, 3, or 4 as an example. The replication defective SARS-CoV-2 RNA genome can further comprising a mutated transcription regulatory sequence (TRS) comprising a nucleic acid sequence of CCGGAT. The replication defective SARS-CoV-2 RNA genome can further comprising a heterologous nucleic acid segment, in certain aspects the heterologous nucleic acid segment is a reporter gene.

Certain embodiments are directed to a producer cell comprising at least one heterologous nucleic acid encoding a ORF3 gene and/or a SARS-CoV-2 gene. In certain aspects, the ORF3 gene and the envelope gene are encoded on the same heterologous nucleic acid. IN other aspects, wherein the ORF3 gene is encoded on a first heterologous nucleic acid and the envelope gene is encoded on a second heterologous nucleic acid.

Certain embodiments are directed to a method for producing non-replicative or single replication SARS-CoV-2 virus comprising, introducing a ΔORF3-E SARS-CoV-2 genomic RNA into ORF3-E SARS-CoV-2 expressing producer cell, wherein the cell produces a non-replicating or single replication SARS-CoV-2 virus containing the ΔORF3-E SARS-CoV-2 genomic RNA.

Certain embodiments are directed to a kit comprising one or more of (i) a replication defective SARS-CoV-2 genome; and/or (ii) a producer cell line that complements the replication defective SARS-CoV-2 genome. In certain aspects, the replication defective SARS-CoV-2 genome is a ΔORF3/Envelope SARS-CoV-2 genome.

Certain embodiments are directed to an expression cassette comprising one or more of: (i) an inducible promoter operably coupled to ORF3 and/or E genes; (ii) an mCherry gene configured to produce a mCherry/E fusion protein upon transcription and translation; (iii) an RNA segment encoding an auto-cleavage site positioned between the mCherry gene and the E gene; (iv) an internal ribosome entry site positioned at the 5′ end of the ORF3 gene. The expression cassette can include a TRE3GS promoter as the inducible promoter. In certain aspects, the auto-cleavage site is a foot-and-mouth disease virus 2A (FMDV 2A) autocleavage site. In certain aspects, the internal ribosome entry site is an encephalomyocarditis virus internal ribosomal entry site (EMCV IRES). The expression cassette can be further comprised in a viral vector. In certain aspects, the viral vector is a lentivirus vector.

Certain embodiments are directed to a stable cell line comprising the expression cassette described above. The expression cassette can be stably integrated into the cell line. In certain aspects, the cell line is a human cell line, for example a Vero E6 cell line.

Certain embodiments are directed to a SARS-COV-2 nucleic acid. In certain aspects the SARS-COV-2 nucleic acids can have at least 90, 95, 98, 99, 99.99, or 100% sequence identity to SEQ ID NO:2, SEQ ID NO:3, or SEQ ID NO:4 or any 10, 20, 30, 40, 50, 100, 200, 300, 400, 500, 600, 700, 800, 1000, 2000, 3000, 4000, 5000, 6000, 7000, 8000, 9000, 10000, 11000 to 29900 consecutive nucleotide segment thereof, including all values and ranges there between. In certain aspects, a SARS-CoV-2 nucleic acid sequence has a sequence that is at least 98% identical to SEQ ID NO:2, SEQ ID NO:3, or SEQ ID NO:4. In certain aspects, a SARS-CoV-2 nucleic acid sequence has a sequence that is 100% identical to SEQ ID NO:2, SEQ ID NO:3, or SEQ ID NO:4.

The term “coronavirus” refers to a virus whose genome is plus-stranded RNA of about 27 kb to about 33 kb in length depending on the particular virus. The virion RNA has a cap at the 5′ end and a poly A tail at the 3′ end. The length of the RNA makes coronaviruses the largest of the RNA virus genomes. Coronavirus RNAs can encode: (1) an RNA-dependent RNA polymerase; (2) N-protein; (3) three envelope glycoproteins; and (4) three non-structural proteins. These coronaviruses infect a variety of mammals and birds. They cause respiratory infections (common), enteric infections (mostly in infants >12 mo.), and possibly neurological syndromes. Coronaviruses are transmitted by aerosols of respiratory secretions. Coronaviruses are exemplified by, but not limited to, human enteric SARS-CoV-2 (GenBank accession number NC 045512.2), coV (ATCC accession #VR-1475), human coV 229E (ATCC accession #VR-740), human coV OC43 (ATCC accession #VR-920), and SARS-coronavirus (Center for Disease Control).

“Nucleic acid” refers to a multimeric compound comprising nucleosides or nucleoside analogs which have nitrogenous heterocyclic bases or base analogs linked together to form a polynucleotide, including conventional RNA, DNA, mixed RNA-DNA, and polymers that are analogs thereof. A nucleic acid “backbone” may be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid may be ribose, deoxyribose, or similar compounds with substitutions, e.g., 2′ methoxy or 2′ halide substitutions. Nitrogenous bases may be conventional bases (A, G, C, T, U), analogs thereof (e.g., inosine or others; see The Biochemistry of the Nucleic Acids 5-36, Adams et al., ed., 11th ed., 1992), derivatives of purines or pyrimidines (e.g., N4-methyl deoxygaunosine, deaza- or aza-purines, deaza- or aza-pyrimidines, pyrimidine bases with substituent groups at the 5 or 6 position, purine bases with a substituent at the 2, 6 or 8 positions, 2-amino-6-methylaminopurine, 06-methylguanine, 4-thio-pyrimidines, 4-amino-pyrimidines, 4-dimethylhydrazine-pyrimidines, and 04-alkyl-pyrimidines; U.S. Pat. No. 5,378,825 and PCT No. WO 93/13121). Nucleic acids may include one or more “abasic” residues where the backbone includes no nitrogenous base for position(s) of the polymer (U.S. Pat. No. 5,585,481). A nucleic acid may comprise only conventional RNA or DNA sugars, bases and linkages, or may include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). Embodiments of oligomers that may affect stability of a hybridization complex include PNA oligomers, oligomers that include 2′-methoxy or 2′-fluoro substituted RNA, or oligomers that affect the overall charge, charge density, or steric associations of a hybridization complex, including oligomers that contain charged linkages (e.g., phosphorothioates) or neutral groups (e.g., methylphosphonates).

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins.

The term “recombinant” refers to an artificial combination of two otherwise separated segments of nucleic acid, e.g., by chemical synthesis or by the manipulation of isolated segments of nucleic acids by genetic engineering techniques.

The term “SARS-CoV-2 replicon particle” refers to a virion or virion-like structural complex incorporating a SARS-CoV-2 replicon.

The term “SARS-CoV-2 reporter virus” refers to a virus that is capable of directing the expression of a sequence(s) or gene(s) of interest. The reporter construct can include a 5′ sequence capable of initiating transcription of a nucleic acid encoding a reporter molecule or protein such as luciferase, fluorescent protein, Neo, SV2 Neo, hygromycin, phleomycin, histidinol, and DHFR. The reporter virus can be used an indicator of infection of a cell by a SARS-CoV-2 virus.

The term “expression vector” refers to a nucleic acid that is capable of directing the expression of a sequence(s) or gene(s) of interest. The vector construct can include a 5′ sequence capable of initiating transcription of a nucleic acid, e.g., all or part of a SARS-CoV-2 virus. The vector may also include nucleic acid molecule(s) to allow for production of virus, a 5′ promoter that is capable of initiating the synthesis of viral RNA in vitro from cDNA, as well as one or more restriction sites, and a polyadenylation sequence. In addition, the constructs may contain selectable markers such as Neo, SV2 Neo, hygromycin, phleomycin, histidinol, and DHFR. Furthermore, the constructs can include plasmid sequences for replication in host cells and other functionalities known in the art. In certain aspects the vector construct is a DNA construct.

“Expression cassette” refers to a nucleic acid segment capable of directing the expression of one or more proteins or nucleic acids.

Stable cell lines are distinct from transiently-transfected cells. Stable cell lines have stable genetic and protein expression characteristics. Transient transfection results in temporary changes to cell lines, which may aid in one-time production of proteins or short-term experiments. This makes transiently-transfected cell lines problematic for studies or procedures that last for several days or more. As such, genetic modifications of cells used for longer studies must be permanent or stable and maintained as the cells propagate in culture.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Generation of single-round infectious ΔORF3-E mNG virion (A) A trans-complementation system for SARS-CoV-2. Vero-ORF3-E cells are electroporated with ΔORF3-E mNG RNA. Trans-complementation produces ΔORF3-E mNG virion (left panel) which can infect naïve Vero E6 cells for only single round (right panel). (B) ΔORF3-E mNG virion genome. Both the full-length mNG SARS-CoV-2 genome (top panel) and the ΔORF3-E mNG virion genome (bottom panel) are shown. The genomic fragment 8 (gF8) of RT-PCR analysis are indicated above both genomes. The ORF3-E deletion junction is indicated. The WT and mutant Transcription Regulatory Sequences (TRS) are also depicted. (C) ORF3-E RNA expression in Vero-ORF3-E cells. Doxycycline (Dox) was used to induce the expression of ORF3-E RNA in Vero-ORF3-E cells. RT-PCR analyses were performed on Vero-ORF3-E cells with or without doxycycline induction as well as on naïve Vero E6 cells. (D) Induction of mCherry expression in Vero-ORF3-E cells. Passage 1 (P1) and P20 of Vero-ORF3-E cells were induced by doxycycline to express mCherry fluorescence. Scale bar, 100 μm. (E) Production of ΔORF3-E mNG virion after electroporation. After electroporating ΔORF3-E mNG RNA into Vero-ORF3-E cells (with doxycycline), infectious titers of ΔORF3-E mNG virion were measured from culture medium. Three sets of repeated experiments are presented with bars representing standard deviations. (F) Negative-staining electron microscopic image of ΔORF3-E mNG virion. Scale bar, 50 nm. (G) Analysis of ΔORF3-E mNG virion infection. Vero E6 or Vero-ORF3-E cells were inoculated with WT mNG SARS-CoV-2 or ΔORF3-E mNG virion. The cells were washed three times with PBS to remove residual input virus. At 48 h post-infection, the supernatants of the infected cells were transferred to fresh Vero E6 or Vero-ORF3-E cells for a second round of infection. The mNG signals from both rounds of infected cells are presented. Scale bar, 100 μm. (H) RT-PCR analysis. Extracellular RNA from the second-round infection from (G) was harvested at 48 h post-infection. Fragment 8 of the viral genome, depicted in (B), was amplified by RT-PCR to confirm the ORF3-E deletion and mNG retention.

FIG. 2. Adaptive mutations to improve the yield of ΔORF3-E mNG virion production. (A) Viral replication kinetics on Vero-ORF3-E cells. Adaptive mutations (D) were selected by continuously passaging the ΔORF3-E virion on Vero-ORF3-E cells for 10 rounds. For comparing the replication kinetics of the passaged viruses, Vero-ORF3-E cells were infected with the P1 or P10 ΔORF3-E virion, ΔORF3-E virion containing an S mutation [ΔORF3-E virion mut-S in (D)], and ΔORF3-E virion all adaptive mutations in nsp1, nsp4, and S [ΔORF3-E virion mut-All in (D)] an MOI of 0.15. WT mNG SARS-CoV-2 was included as a control. Viral titers in culture supernatants are presented. ANOVA with multiple comparison correction test were performed with *, P<0.05; **, P<0.01. (B) mNG-positive cells at 24 and 48 h post-infection from (A). (C) RT-PCR analysis for single-round infection. For confirming the P10 ΔORF3-E virion remains infectious for only a single-round on Vero cells, Vero E6 or Vero-ORF3-E cells were infected with WT mNG SARS-CoV-2 or P10 ΔORF3-E mNG virion for two rounds as described in FIG. 1G. Viral RNAs were extracted from the second-round culture fluids and analyzed by RT-PCR. The RT-PCR product, representing genomic fragment 8 (gF8), is indicated in FIG. 1B. (D) Adaptive mutations. Three mutations were identified from whole genome sequencing of P10 ΔORF3-E mNG virion. No mutation was found in the P1 ΔORF3-E mNG virion.

FIG. 3. Safety characterization of ΔORF3-E mNG virion in animal models. (A) Hamster experimental schedule. Four- to five-week-old male Syrian golden hamsters were intranasally (I.N.) inoculated with 10⁵ TCID₅₀ of WT SARS-CoV-2, 10⁶ TCID₅₀ ΔORF3-E mNG virion, or PBS control. Hamsters were monitored for weight loss, disease symptom, and viral RNA load. (B) Hamster weight change (n=9). (C) Hamster disease symptoms (n=9). (D) Hamster nasal wash viral RNA level (n=5). (E) Hamster oral swab viral RNA level (n=5). (F) Viral RNA loads in hamster trachea and lung at day 2 post-infection (n=5). Limit of detection, L.O.D. (G) Mouse experimental schedule. Nine-week-old K18-hACE2 mice were inoculated with WT SARS-CoV-2 or ΔORF3-E mNG virion via the intranasal (I.N.) or intracranial C.) route. (H) Mouse weight loss after I.N. infection. Mice were intranasally inoculated with 2.5×10³ TCID₅₀ of WT SARS-CoV-2, 3×10⁵ TCID₅₀ of ΔORF3-E mNG virion, or PBS mock. Body weights were normalized to the initial body weight. The means of WT SARS-CoV-2 (n=9), ΔORF3-E mNG virion (n=4), and mock (n=4) groups are indicated. Error bars indicate the standard deviation. Kruskal-Wallis ANOVA with Dunn's multiple comparison post-test was performed to evaluate the statistical significance among groups. *, P<0.05; **, P<0.01; ***, P<0.001; ****, P<0.0001. (I) Mouse survival after I.N. infection. Log-rank (Mantel-Cox) was performed to evaluate the statistical significance among groups. (J) Mouse weight loss after I.C. infection. The mean weight values from groups WT SARS-CoV-2 500 TCID₅₀ (n=4), 50 TCID₅₀ (n=5), 5 TCID₅₀ (n=5), and 1 TCID₅₀ (n=5) and 6×10³ TCID₅₀ ΔORF3-E mNG virion (n=4) are indicated. Kruskal-Wallis ANOVA with Dunn's multiple comparison post-test was performed to evaluate the statistical significance between groups. *, P<0.05; **, P<0.01; ***, P<0.001; ****, P<0.0001. (K) Mouse survival after I.C. infection. Log-rank (Mantel-Cox) was performed to evaluate the statistical significance among groups.

FIG. 4. ΔORF3-E mNG virion-based high-throughput neutralization and antiviral testing. (A) Assay scheme in a 96-well format. (B) Correlation analysis of NT50 values between the ΔORF3-E mNG virion assay and plaque-reduction neutralization test (PRNT). The Pearson correlation efficiency R² and P value (two-tailed) are indicated. (C) Neutralization curves. Representative curves are presented for one negative and three positive sera. The means and standard deviations from two independent experiments are shown. (D) EC 50 of human mAb14 against ΔORF3-E mNG virion infecting Vero CCL81 cells. The mean±standard deviations from four independent experiments are indicated. (E) EC₅₀ of Remdesivir against ΔORF3-E mNG virion infecting A549-hACE2 cells. (F) EC 50 of Remdesivir against ΔORF3-E mNG virion on Vero CCL81 cells. For (E) and (F), the mean±standard deviations from three independent experiments are indicated. The four-parameter dose-response curve was fitted using the nonlinear regression method.

FIG. 5. Construction of Vero-ORF3-E cell lines. (A) Construction of a lentiviral transfer plasmid encoding mCherry, ORF3, and E protein. The sequence of FMDV 2A and its translational break position is indicated by an arrow. (B) Merged mCherry (red) and nuclei (blue) images of 3 selected clones of Vero-ORF3-E cell lines. Nuclei were stained with Hoechst 33342. Doxycycline induction is indicated. (C) mCherry expression in doxycycline-induced cells. mCherry-positive cells were quantified using a plate reader. The percentages of mCherry positive cells are presented. The results are presented as means and standard deviations from six replicates, and more than 10⁵ cells were counted for each clone. Clone 1 was used in the rest of this study.

FIG. 6. Single-round infection of ΔORF3-E mNG virion. (A) Calu-3 and A549-hACE2 cells (MOI of 1; viral titers determined on Vero-ORF3-E cells) were infected with mNG SARS-CoV-2 or ΔORF3-E mNG virion for 2 h, after which the cells were washed and cultured in fresh medium. At day 2 post-infection, supernatants of the infected cells were transferred to infect naïve Calu-3 and A549-hACE2 for the second round. Fluorescence and phase contrast images for the first and second sound infected cells are presented. (B) RT-PCR analysis of viral RNA. Extracellular RNAs from the second round of infection from (A) were harvested at day 2 post-infection and subjected to RT-PCR analysis of viral RNA.

FIG. 7. No WT mNG SARS-CoV-2 production from the trans-complementation system. WT mNG SARS-CoV-2 and P10 ΔORF3-E mNG virion (derived from 10 rounds of passaging of ΔORF3-E mNG virion on Vero-ORF3-E cells) were used to infect naïve Vero E6 cells for two rounds as described in FIG. 1G. (A) Fluorescence and phase contrast images of infected cells are presented for both the first and second rounds of infections. (B) RT-PCR analysis of viral RNA extracted from the culture fluids from the second-round infected cells.

FIG. 8. Selection of ΔORF3-E mNG virion capable of inefficiently infecting Vero E6 cells for more than one round. Four independently selected P5 ΔORF3-E mNG virions (generated from five rounds of passaging ΔORF3-E mNG virion on Vero-ORF3-E cells) were used to infect naïve Vero E6 cells for two rounds as described in FIG. 1G. The P5 ΔORF3-E mNG virion-infected Vero cells were analyzed for mNG signals under a fluorescence microscope (A). The extracellular RNA from the second-round infected cells were analyzed by RT-PCR for viral RNA (B). The replication kinetics of WT mNG SARS-CoV-2 and Selection IV P5 ΔORF3-E (S-IV-P5) mNG virion were compared on Vero E6 cells (C). The cells were inoculated at an MOI of 0.001. Limit of detection, L.O.D. Adaptive mutations were identified from the S-IV-P5 mNG virion (D). The T130N mutation from the M protein was engineered to ΔORF3-E mNG virion. The resulting ΔORF3-E mNG M T130N virion was used to infect naïve Vero E6 cells for two rounds. Fluorescence and phase contrast images of the infected cells are shown (E). Sequence alignment shows that the M protein from SARS-CoV and SARS-CoV-2 shares the same T130 residue (F). Arrow indicates the T130 residue of SARS-CoV-2.

FIG. 9. No improvement of viral replication of Selection IV ΔORF3-E (S-IV-P5) mNG virion after 10 rounds of culturing on Vero E6 cells. S-IV-P5 mNG virion was continuously passaged on Vero E6 cells for 10 rounds. The P2 and P10 S-IV-P5 mNG virions were used to infect naïve Vero E6 cells at an MOI of 0.001. The mNG-positive cells (A) and the growth kinetics of the P2 and P10 S-IV-P5 mNG virions (B) were compared. We did not use the P1 S-IV-P5 mNG virion in this experiment because the P1 stock retained some carryover virions derived from the Vero-ORFS-E trans-complementation culture.

FIG. 10. Safety characterization of S-IV-P5 mNG virion in hamsters. The weight change (A) and disease symptoms (B) of hamsters (n=5) that were intranasally infected with 5,000 TCID 50 of S-IV-P5 mNG virion. The high titer of S-IV-P5 mNG virion for this experiment was prepared by amplifying the virion on Vero-ORF3-E cells.

FIG. 11. Safety analysis of S-IV-P5 mNG virion in K18-hACE transgenic mice. Nine-week-old K18-hACE2 mice were inoculated with S-IV-P5 mNG virion via the intranasal (I.N.) or intracranial (I.C.) route. Mouse body weight and survival were monitored for 14 days. (A) Mouse weight loss after I.N. infection. Mice were infected with 2,500 TCID₅₀ of S-IV-P5 mNG virion (n=4) or PBS mock (n=4) via the I.N. route. The mean±standard deviations are indicated. (B) Mouse survival after I.N. infection. (C) Mouse weight loss after I.C. infection. Mouse were inoculated with 500 TCID₅₀ of S-IV-P5 mNG virion (n=4) via the I.C. route. The mean±standard deviations are indicated. (D) Mouse survival after I.C. infection. The high titer of S-IV-P5 mNG virion for this experiment was prepared by amplifying the virion on Vero-ORF3-E cells.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

The inventors generated and characterized a trans-complementation system for SARS-CoV-2. The system produced a high yield of single-round infectious ΔORF3-E mNG virion that could be used for neutralization and antiviral testing. Both the single-round virion (when infecting wild-type cells) and the multi-round system (when infecting complementing cells such as Vero-ORF3-E) can be used for diagnosis, neutralization, and antiviral testing. In certain aspects, an mNG reporter was introduced into the ΔORF3-E virion to be an indicator of viral replication. Depending on research needs, other reporter genes, such as luciferase, GFP, etc, could be engineered into the system. A reliable high-throughput neutralization assay is important for COVID-19 vaccine evaluation and for studying the kinetics of neutralizing antibody levels in post-vaccinated and naturally infected people (4, 21, 22). Three types of cell-based high-throughput neutralization assays currently are available: (i) pseudovirus assay, which expresses SARS-CoV-2 S protein alone, can be performed at BSL-2 laboratories (23, 24); (ii) a reporter SARS-CoV-2 assay, which must be performed at BSL-3 laboratories, represents authentic viral infection (3, 5, 6, 25); (iii) bona fide fully infectious SARS-CoV-2 by focus reduction neutralization test (23). The ΔORF3-E mNG virion combines the advantages of each assay type by recapitulating the authentic viral infection for a single round, thus qualifying its use at BSL2 laboratories. The ΔORF3-E mNG virion can be readily adapted to investigate vaccine-elicited neutralization against newly emerged SARS-CoV-2 isolates, such as the rapidly spreading United Kingdom and South African strains (26, 27), by swapping or mutating the S gene.

The trans-complementation system also can be used for high-throughput antiviral screening of large compound libraries. Infection of normal cells with ΔORF3-E mNG virions allows for screening of inhibitors of virus entry, translation, and RNA replication, but not virion assembly/release. In contrast, infection of Vero-ORF3-E cells with ΔORF3-E mNG virion can be used to identify inhibitors of all steps of SARS-CoV-2 infection cycle, including virion assembly and release; this system also allows for resistance selection against inhibitors for mode-of-action studies. In addition, the single-round ΔORF3-E virion could be developed as a safe vaccine platform.

The results support that the trans-complementation system can be performed safely in BSL-2 laboratories. (i) The system produced single-round infectious ΔORF3-E mNG virion that does not infect normal cells for multiple rounds. (ii) The system did not produce WT virus, even after multiple independent selections. (iii) Although an adaptive mutation in M protein was selected to confer virion for multi-round infection on normal cells (i.e., S-IV-P5), the replication level of S-IV-P5 was barely detectable, with infectious titers >10⁵-fold lower than the WT SARS-CoV-2. The molecular mechanism of how S-IV-P5 could infect cells for multiple rounds without ORF3 and E proteins remains to be defined. Previous studies showed that deletion of ORF3 and E genes was lethal for SARS-CoV (28). (iv) Continuous culturing of the S-IV-P5 virion on naïve Vero cells did not improve viral replication. (v) When hamsters and K18-hACE2 mice were infected with the highest possible doses, neither ΔORF3-E mNG virion nor S-IV-P5 virion caused morbidity or mortality. Even after intracranial infection with the highest possible dose, neither virions caused disease or death in the highly susceptible K18-hACE2 mice. To further improve the safety of the system, we could delete more accessory ORFs from the ΔORF3-E mNG RNA as accessory proteins are not essential for viral replication (1).

The current examples use of Vero E6 cells as a representative cell line for constructing a Vero-ORF3-E cell line. When propagated on Vero E6 cells, SARS-CoV-2 could accumulate deletions at the furin cleavage site in the S protein (29, 30). The furin cleavage deletion affects the neutralization susceptibility of SARS-CoV-2 and possibly the route of entry into cells (31). Although furin cleavage deletions were not observed when ΔORF3-E mNG virion was passaged on the Vero-ORF3-E cells, this possibility can be minimized or eliminated by using other cell lines, such as, but not limited to A549-hACE2 or Vero-TMPRSS2-hACE2 cells.

### Complementing Cells

Cell lines or primary cells can be transformed with an expression cassette to produce a cell or cell line of the invention resulting in a trans-complementing cell line(s). A precursor to the trans-complementing cell line can be selected from any mammalian species, such as human cell types, including without limitation, cells such as primary cells isolated from various human tissues, e.g., human tonsil or umbilical cord cells; cell lines such as HeLa, Vero, A549 and/or HKB cells or other human cell lines. Other mammalian species cells are also useful, for example, primate cells, rodent cells or other cells commonly used in biological laboratories. The selection of the mammalian species providing the cells is not a limitation of this invention; nor is the type of mammalian cell, i.e., fibroblast, hepatocyte, tumor cell, etc.

Suitably, the target cells are transformed with a nucleic acid, e.g. an expression cassette, comprising nucleic acid sequences encoding coronavirus ORF3 and E under the control of a heterologous promoter.

The DNA sequences encoding the coronavirus genes useful in this invention may be selected from among any known coronavirus type, including the presently identified SARS-CoV-2. Similarly, coronaviruses known to infect other animals may supply the gene sequences. The selection of the coronavirus type for each gene sequence does not limit this invention. The sequences for a number of coronavirus serotypes are available from Genbank. A variety of coronavirus strains are available from the ATCC, or are available by request from a variety of commercial and institutional sources. In the following examples of sequences are those from a representative coronavirus, SARS-CoV-2.

By “nucleic acid that expresses the ORF3 gene product,” it is meant any adenovirus gene encoding ORF3 protein (including proteins that are 85, 90, 91, 92, 93, 94, 95, 96, 97, 98, 99% or more identical in amino acid sequence) or any functional ORF3 polypeptide segment thereof. Similarly included are any alleles or other modifications of the ORF3 gene or functional portion. Such modifications may be deliberately introduced by resort to conventional genetic engineering or mutagenic techniques to enhance the ORF3 expression or function in some manner, as well as naturally occurring allelic variants thereof. The nucleic acid sequence may be modified to reduce the identity.

By “nucleic acid that expresses the envelope or E gene product,” it is meant any coronavirus gene encoding E (including proteins that are 85, 90, 91, 92, 93, 94, 95, 96, 97, 98, 99% or more identical in amino acid sequence) or any functional E portion. Similarly included are any alleles or other modifications of the E gene or functional portion. Such modifications may be deliberately introduced by resort to conventional genetic engineering or mutagenic techniques to enhance the E expression or function in some manner, as well as naturally occurring allelic variants thereof.

The nucleic acid molecule carrying the ORF3 and E genes may be in any form which transfers these components to the host cell. Most suitably, these sequences are contained within an expression cassette or an expression vector. An “expression cassette” includes a polynucleotide that includes all elements for expression, such as a promoter and a poly-adenylation site. An “expression vector” includes, without limitation, any genetic element, such as a plasmid, phage, transposon, cosmid, chromosome, virus, virion, etc. that include elements for propagation, insertion, or other functions not directly related to expression of a coding region. In one aspect, the nucleic acid molecule is a plasmid carrying coronavirus ORF3 and/or E sequences under the control of a heterologous promoter, that is a promoter that is not the typical promoter used by coronavirus to express the ORF3 and/or E genes. In certain aspects, the promoter can be an inducible promoter, such as, but not limited to a TRE3GS doxycycline inducible promoter.

The nucleic acid molecule may contain other non-viral sequences, such as those encoding certain selectable reporters or marker genes, e.g., sequences encoding hygromycin or purimycin, or the neomycin resistance gene for G418 selection, among others. The molecule may further contain other components.

Conventional techniques may be utilized for construction of the nucleic acid molecules of the invention. See, generally, Sambrook et al., Molecular Cloning: A Laboratory Manual, Cold Spring Harbor Laboratories, Cold Spring Harbor, N.Y.

Once the desired nucleic acid molecule is engineered, it may be transferred to the target cell by any suitable method. Such methods include, for example, transfection, electroporation, liposome delivery, membrane fusion techniques, high velocity DNA-coated pellets, viral infection and protoplast fusion. Thereafter, cells are cultured according to standard methods and, optionally, seeded in media containing an antibiotic to select for cells containing the cells expressing the resistance gene. After a period of selection, the resistant colonies are isolated, expanded, and screened for E1 expression. See, Sambrook et al., cited above.

Promoters and Enhancers—In order for the expression cassette to effect expression of complementing components, the nucleic acid encoding regions will be under the transcriptional control of a promoter. A “promoter” is a control sequence that is a region of a nucleic acid sequence at which initiation and rate of transcription are controlled. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

Any promoter known to those of ordinary skill in the art that would be active in a complementing cell is contemplated as a promoter that can be applied in the methods and compositions of the present invention. One of ordinary skill in the art would be familiar with the numerous types of promoters that can be applied in the present methods and compositions. In certain embodiments, for example, the promoter is a constitutive promoter, an inducible promoter, or a repressible promoter. Examples of promoters include inducible promoters such as the TRE3GS promoter.

An endogenous promoter is one that is naturally associated with a gene or sequence. Certain advantages are gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™ (see U.S. Pat. Nos. 4,683,202 and 5,928,906).

Naturally, it will be important to employ a promoter and/or enhancer that effectively directs the expression of the DNA segment in the complementing cell. Those of skill in the art of molecular biology generally understand the use of promoters, enhancers, and cell type combinations for protein expression, for example, see Sambrook et al. (2001).

The particular promoter that is employed to control the expression of the nucleic acid of interest is not believed to be critical, so long as it is capable of expressing the polynucleotide in the targeted cell at sufficient levels. Thus, where a human cell is targeted, it is preferable to position the polynucleotide coding region adjacent to and under the control of a promoter that is capable of being expressed in a human cell. Generally speaking, such a promoter might include either a human or viral promoter.

In various embodiments, the TRE3GS inducible promoter, the human cytomegalovirus (CMV) immediate early gene promoter, the SV40 early promoter and the Rous sarcoma virus long terminal repeat can be used. The use of other viral or mammalian cellular or bacterial phage promoters well-known in the art to achieve expression of polynucleotides is contemplated as well, provided that the levels of expression are sufficient to produce an complementing cell line. Additional examples of promoters/elements that may be employed, in the context of the present invention include the following, which is not intended to be exhaustive of all the possible promoter and enhancer elements, but, merely, to be exemplary thereof.

Immunoglobulin Heavy Chain (Banerji et al., 1983; Gilles et al., 1983; Grosschedl et al., 1985; Atchinson et al., 1986, 1987; Imler et al., 1987; Weinberger et al., 1984; Kiledjian et al., 1988; Porton et al.; 1990); Immunoglobulin Light Chain (Queen et al., 1983; Picard et al., 1984); T Cell Receptor (Luria et al., 1987; Winoto et al., 1989; Redondo et al.; 1990); HLA DQ a and/or DQ βSullivan et al., 1987); β Interferon (Goodbourn et al., 1986; Fujita et al., 1987; Goodbourn et al., 1988); Interleukin-2 (Greene et al., 1989); Interleukin-2 Receptor (Greene et al., 1989; Lin et al., 1990); WIC Class II (Koch et al., 1989); WIC Class II HLA-DRa (Sherman et al., 1989); (3-Actin (Kawamoto et al., 1988; Ng et al.; 1989); Muscle Creatine Kinase (MCK) (Jaynes et al., 1988; Horlick et al., 1989; Johnson et al., 1989); Prealbumin (Transthyretin) (Costa et al., 1988); Elastase I (Omitz et al., 1987); Metallothionein (MTII) (Karin et al., 1987; Culotta et al., 1989); Collagenase (Pinkert et al., 1987; Angel et al., 1987); Albumin (Pinkert et al., 1987; Tronche et al., 1989, 1990); α-Fetoprotein (Godbout et al., 1988; Campere et al., 1989); t-Globin (Bodine et al., 1987; Perez-Stable et al., 1990); β-Globin (Trudel et al., 1987); c-fos (Cohen et al., 1987); c-HA-ras (Triesman, 1986; Deschamps et al., 1985); Insulin (Edlund et al., 1985); Neural Cell Adhesion Molecule (NCAM) (Hirsh et al., 1990); α1-Antitrypsin (Latimer et al., 1990); H2B (TH2B) Histone (Hwang et al., 1990); Mouse and/or Type I Collagen (Ripe et al., 1989); Glucose-Regulated Proteins (GRP94 and GRP78) (Chang et al., 1989); Rat Growth Hormone (Larsen et al., 1986); Human Serum Amyloid A (SAA) (Edbrooke et al., 1989); Troponin I (TN I) (Yutzey et al., 1989); Platelet-Derived Growth Factor (PDGF) (Pech et al., 1989); Duchenne Muscular Dystrophy (Klamut et al., 1990); SV40 (Banerji et al., 1981; Moreau et al., 1981; Sleigh et al., 1985; Firak et al., 1986; Herr et al., 1986; Imbra et al., 1986; Kadesch et al., 1986; Wang et al., 1986; Ondek et al., 1987; Kuhl et al., 1987; Schaffner et al., 1988); Polyoma (Swartzendruber et al., 1975; Vasseur et al., 1980; Katinka et al., 1980, 1981; Tyndell et al., 1981; Dandolo et al., 1983; de Villiers et al., 1984; Hen et al., 1986; Satake et al., 1988; Campbell and/or Villarreal, 1988); Retroviruses (Kriegler et al., 1982, 1983; Levinson et al., 1982; Kriegler et al., 1983, 1984a, b, 1988; Bosze et al., 1986; Miksicek et al., 1986; Celander et al., 1987; Thiesen et al., 1988; Celander et al., 1988; Choi et al., 1988; Reisman et al., 1989); Papilloma Virus (Campo et al., 1983; Lusky et al., 1983; Wilkie, 1983; Spalholz et al., 1985; Lusky et al., 1986; Cripe et al., 1987; Gloss et al., 1987; Hirochika et al., 1987; Stephens et al., 1987); Hepatitis B Virus (Bulla et al., 1986; Jameel et al., 1986; Shaul et al., 1987; Spandau et al., 1988; Vannice et al., 1988); Human Immunodeficiency Virus (Muesing et al., 1987; Hauber et al., 1988; Jakobovits et al., 1988; Feng et al., 1988; Takebe et al., 1988; Rosen et al., 1988; Berkhout et al., 1989; Laspia et al., 1989; Sharp et al., 1989; Braddock et al., 1989); Cytomegalovirus (CMV) (Weber et al., 1984; Boshart et al., 1985; Foecking et al., 1986); Gibbon Ape Leukemia Virus (Holbrook et al., 1987; Quinn et al., 1989).

Enhancers were originally detected as genetic elements that increased transcription from a promoter located at a distant position on the same molecule of DNA. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have very similar modular organization. Additionally, any promoter/enhancer combination (as per the Eukaryotic Promoter Data Base EPDB) could also be used to drive expression of a gene. Further selection of a promoter that is regulated in response to specific physiologic signals can permit inducible expression of a construct. For example, with the polynucleotide under the control of the human PAI-1 promoter, expression is inducible by tumor necrosis factor. Examples of inducible elements, which are regions of a nucleic acid sequence that can be activated in response to a specific stimulus include (Element/Inducer): MT II/Phorbol Ester (TFA) or Heavy metals (Palmiter et al., 1982; Haslinger et al., 1985; Searle et al., 1985; Stuart et al., 1985; Imagawa et al., 1987, Karin et al., 1987; Angel et al., 1987b; McNeall et al., 1989); MMTV (mouse mammary tumor virus)/Glucocorticoids (Huang et al., 1981; Lee et al., 1981; Majors et al., 1983; Chandler et al., 1983; Ponta et al., 1985; Sakai et al., 1988); β-Interferon/poly(rI)x or poly(rc) (Tavernier et al., 1983); Adenovirus 5 E2/E1A (Imperiale et al., 1984); Collagenase/Phorbol Ester (TPA) (Angel et al., 1987a); Stromelysin/Phorbol Ester (TPA) (Angel et al., 1987b); SV40/Phorbol Ester (TPA) (Angel et al., 1987b); Murine MX Gene/Interferon, Newcastle Disease Virus (Hug et al., 1988); GRP78 Gene/A23187 (Resendez et al., 1988); α-2-Macroglobulin/IL-6 (Kunz et al., 1989); Vimentin/Serum (Rittling et al., 1989); MEW Class I Gene H-2κb/Interferon (Blanar et al., 1989); HSP70/E1A, SV40 Large T Antigen (Taylor et al., 1989, 1990a, 1990b); Proliferin/Phorbol Ester-TPA (Mordacq et al., 1989); Tumor Necrosis Factor/PMA (Hensel et al., 1989); and Thyroid Stimulating Hormone a Gene/Thyroid Hormone (Chatterjee et al., 1989).

### Use of Complementing Cells in Production of ΔORF3/E Coronavirus

The complementing cells of the invention are useful for a variety of purposes. Typically, the cells are used in packaging recombinant virus (i.e., viral particles) from defective vectors and in production of defective viruses.

The cells of the invention which express ORF3 and E are suitable for use in packaging recombinant virus from ORF3/E defective vectors or viral genomes. Further, these cells are anticipated to be useful in producing recombinant virus from other coronavirus.

### Packaging of Coronavirus ΔORF3/E Nucleic Acids

In certain embodiments, this method of the invention involves packaging of an ORF3/E-deleted vector or genome containing a heterologous nucleic acid segment into an coronavirus particle useful for delivery of the heterologous nucleic acid to a host cell. In certain aspects, the ORF3/E-deleted vector or genome contains all other coronavirus genes necessary to produce and package an coronavirus particle which replicates only in the presence of complementing ORF3/E proteins, e.g., such as are supplied by cell line of the invention. The vector contains defects in the ORF3 and E sequences, and most desirably, is deleted of all or most of these gene sequences.

### Coronavirus Elements

Coronaviruses (order Nidovirales, family Coronaviridae) are a diverse group of enveloped, positive-stranded RNA viruses. The coronavirus genome, approximately 27-32 Kb in length, is the largest found in any of the RNA viruses. Large Spike (S) glycoproteins protrude from the virus particle giving coronaviruses a distinctive corona-like appearance when visualized by electron microscopy. Coronaviruses infect a wide variety of species, including canine, feline, porcine, murine, bovine, avian and human (Holmes, et al., 1996, Coronaviridae: the viruses and their replication, p. 1075-1094, Fields Virology, Lippincott-Raven, Philadelphia, Pa.). However, the natural host range of each coronavirus strain is narrow, typically consisting of a single species. Coronaviruses typically bind to target cells through Spike-receptor interactions and enter cells by receptor mediated endocytosis or fusion with the plasma membrane (Holmes, et al., 1996, supra).

Upon entry into susceptible cells, the open reading frame (ORF) nearest the 5′ terminus of the coronavirus genome is translated into a large polyprotein. This polyprotein is autocatalytically cleaved by viral-encoded proteases, to yield multiple proteins that together serve as a virus-specific, RNA-dependent RNA polymerase (RdRP). The RdRP replicates the viral genome and generates 3′ coterminal nested subgenomic RNAs. Subgenomic RNAs include capped, polyadenylated RNAs that serve as mRNAs, and antisense subgenomic RNAs complementary to mRNAs. In one embodiment, each of the subgenomic RNA molecules shares the same short leader sequence fused to the body of each gene at conserved sequence elements known as intergenic sequences (IGS), transcriptional regulating sequences (TRS) or transcription activation sequences. It has been controversial as to whether the nested subgenomic RNAs are generated during positive or negative strand synthesis; however, recent work favors the model of discontinuous transcription during minus strand synthesis (Sawicki, et al., 1995, Adv. Exp. Med. Biol. 380:499-506; Sawicki and Sawicki Adv. Expt. Biol. 1998, 440:215).

A SARS-CoV-2 reference sequence can be found in GenBank accession NC 045512.2 as of Mar. 2, 2020 (SEQ ID NO:1), which is a representative non-limiting coronavirus sequence, other coronavirus variants having 90, 91, 92, 93, 94, 95, 96, 97, 98, 99% identity as determine by a BLAST comparison are also contemplated and can be engineered in a similar fashion as described herein. This particular sequence is a 29903 bp ss-RNA and is referred to as the Wuhan seafood market pneumonia virus isolate Wuhan-Hu-1. The virus is Severe acute respiratory syndrome coronavirus 2 (SARS-CoV-2) with the taxonomy of Viruses; Riboviria; Nidovirales; Cornidovirineae; Coronaviridae; Orthocoronavirinae; Betacoronavirus; Sarbecovirus. (Wu et al. “A novel coronavirus associated with a respiratory disease in Wuhan of Hubei province, China” Unpublished; NCBI Genome Project, Direct Submission, Submitted (17 Jan. 2020) National Center for Biotechnology Information, NIH, Bethesda, MD 20894, USA; Wu et al. Direct Submission, Submitted (5 Jan. 2020) Shanghai Public Health Clinical Center and School of Public Health, Fudan University, Shanghai, China).

The genome of SARS-CoV-2, with reference to SEQ ID NO:1, includes (1) a 5′UTR 1-265), (2) Orf1ab gene (266-21555), S gene encoding a spike protein (21563 . . . 25384), ORF3a gene (25393 . . . 26220), E gene encoding E protein (26245 . . . 26472), M gene (26523 . . . 27191), ORF6 gene (27202 . . . 27387), ORF7a gene (27394 . . . 27759), ORF7b gene (27756 . . . 27887), ORF8 gene (27894 . . . 28259), N gene (28274 . . . 29533), ORF10 gene (29558 . . . 29674), and 3′UTR (29675 . . . 29903). In certain aspects, ORF7 is substituted by a nucleic acid encoding a reporter protein.

### Transgene

The composition of the transgene sequence will depend upon the use to which the resulting virus will be put. For example, one type of transgene sequence includes a reporter sequence, which upon expression produces a detectable signal. Such reporter sequences include without limitation, DNA sequences encoding β-lactamase, β-galactosidase (LacZ), alkaline phosphatase, thymidine kinase, fluorescent protein (such as green fluorescent protein (GFP)), chloramphenicol acetyltransferase (CAT), and/or luciferase, for example. Methods of detecting reporters are well known. Examples of reporter proteins, e.g., luminescent or marker proteins, that can be used in embodiments of the invention include, but are not limited to, Aequorin, firefly luciferase, Renilla luciferase, red luciferase, luxAB, and nanoluciferase. Examples of chemiluminescent protein or marker protein include β-galactosidase, horseradish peroxidase (HRP), and alkaline phosphatase. Examples of fluorescent protein or marker protein include, but are not limited to, mNeonGreen, TagBFP, Azurite, EBFP2, mKalama1, Sirius, Sapphire, T-Sapphire, ECFP, Cerulean, SCFP3A, mTurquoise, monomeric Midoriishi-Cyan, TagCFP, mTFP1, EGFP, Emerald, Superfolder GFP, Monomeric Azami Green, TagGFP2, mUKG, mWasabi, EYFP, Citrine, Venus, SYFP2, TagYFP, Monomeric Kusabira-Orange, mKOK, mKO2, mOrange, mOrange2, mRaspberry, mCherry, dsRed, mStrawberry, mTangerine, tdTomato, TagRFP, TagRFP-T, mApple, mRuby, mPlum, HcRed-Tandem, mKate2, mNeptune, NirFP, TagRFP657, IFP1.4, iRFP, mKeima Red, LSS-mKate1, LSS-mKate2, PA-GFP, PAmCherry 1, PATagRFP, Kaede (green), Kaede (red), KikGR1 (green), KikGR1 (red), PS-CFP2, PS-CFP2, mEos2 (green), mEos2 (red), PSmOrange, or Dronpa.

### Examples

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Results

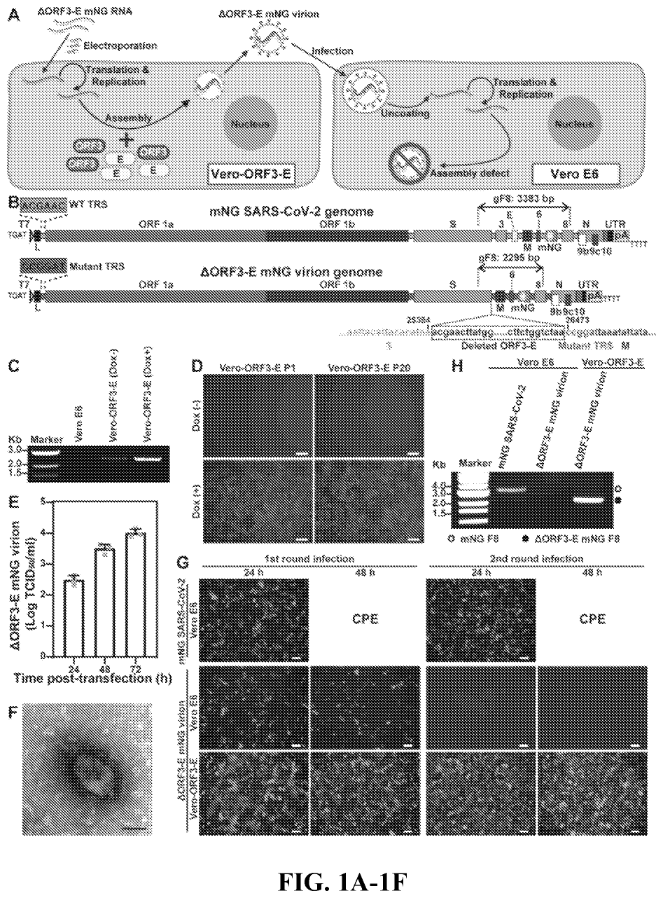

A single-round infectious SARS-CoV-2 system. FIG. 1A depicts the trans-complementation system to produce single-round infectious SARS-CoV-2. The system contains two components: (i) a viral RNA containing a mNeonGreen (mNG) reporter gene and a deletion of ORF3 and E genes (ΔORF3-E; FIG. 1B) and (ii) a Vero E6 cell line expressing the ORF3 and E proteins under a doxycycline inducible promoter (Vero-ORF3-E; FIG. 1C-D). Upon electroporation of ΔORF3-E RNA into Vero-ORF3-E cells and addition of doxycycline, trans-complementation enables production of virions that can continuously infect and amplify on Vero-ORF3-E cells; however, these virions can only infect normal cells for a single round due to the lack of ORF3 and E proteins (FIG. 1A).

Our trans-complementation system is engineered with several safeguards to eliminate wild-type (WT) SARS-CoV-2 production. Besides the ORF3-E deletion, the ΔORF3-E viral RNA contained two additional modifications. (i) The transcription regulatory sequence (TRS) of ΔORF3-E RNA was mutated from the WT ACGAAC to CCGGAT (mutant nucleotides underlined; FIG. 1B). Recombination between the TRS-mutated ΔORF3-E RNA with inadvertently contaminating viral RNA would not produce replicative virus (14, 15). (ii) An mNG gene was engineered at ORF7 of ΔORF3-E RNA to facilitate the detection of viral replication (FIG. 1B). The trans-complementing Vero-ORF3-E cell lines were produced by transducing Vero E6 cells with a lentivirus encoding the following elements (FIG. S1A): a TRE3GS promoter that allows doxycycline to induce ORF3 and E protein expression (FIG. 1C-D and SIB), an mCherry gene that facilitates selection of cell lines with high levels of protein expression (FIG. S1C), a foot-and-mouth disease virus 2A (FMDV 2A) autocleavage site that enables translation of individual mCherry and viral E protein, and an encephalomyocarditis virus internal ribosomal entry site (EMCV IRES) that bicistronically translates viral ORF3 protein. The above design eliminated overlapping sequences between the ORF3-E mRNA and ΔORF3-E viral RNA, thus minimizing homologous recombination during trans-complementation. The Vero-ORF3-E cell line stably expressed the engineered proteins after 20 rounds of passaging, as indicated by the mCherry reporter (FIG. 1D).

Electroporation of ΔORF3-E mNG RNA into doxycycline-induced Vero-ORF3-E cells produced virions of 10⁴ median Tissue Culture Infectious Dose (TCID₅₀)/ml (FIG. 1E). The ΔORF3-E mNG virion exhibited a diameter of ˜91 nm under negative staining electron microscopy (FIG. 1F). The virion produced in the supernatant could infect Vero-ORF3-E cells for multiple rounds, but for only one round on naïve Vero E6 (FIG. 1G-H), Calu-3, or hACE2-expressing A549 cells (A549-hACE2; FIG. S2). As controls, WT mNG SARS-CoV-2 could infect cells for multiple rounds (FIG. S2). These results indicate that the trans-complementation system produces virions that can only infect WT cells for single round.

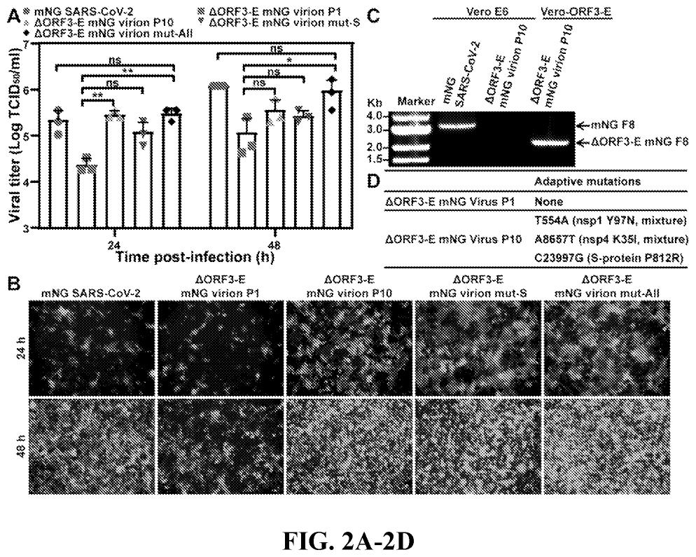

Adaptive mutations to improve virion production. To improve the efficiency of the trans-complementation platform, we continuously propagated ΔORF3-E mNG virions on Vero-ORF3-E cells for 10 passages β-4 days per passage) to select for adaptive mutations. The P10 virion replicated to higher titers than the P1 virion on Vero-ORF3-E cells (FIG. 2A), retained the mNG reporter (FIG. 2B-C), and still infected parental Vero cells for only single round (FIG. S3). Whole genome sequencing of the P10 virion revealed three mutations in nsp1, nsp4, and spike genes (FIG. 2D). Engineering of these mutations into ΔORF3-E mNG RNA showed that all three were required to enhance the trans-complementation efficiency, producing 10⁶ TCID₅₀/ml of virions (FIG. 2A-B). These results indicate that (i) adaptive mutations can be selected to improve the yield of single-round virions and (ii) WT virus is not produced from the trans-complementation system.

Exclusion of WT SARS-CoV-2 production. To confirm that no WT SARS-CoV-2 is inadvertently produced during trans-complementation, we performed four additional selections by passaging ΔORF3-E mNG virions on Vero-ORF3-E cells for five rounds. The P5 virions from selections I-III could only infect Vero cells for single round (FIG. S4A-B). Unexpectedly, selection IV produced P5 (S-IV-P5) virions that could infect parental Vero E6 cells for more than one round, though at a barely detectable level of ˜10² TCID₅₀/ml, which was >10⁵-fold lower than the WT mNG SARS-CoV-2 (FIG. S4C). Full-genome sequencing revealed that the S-IV-P5 genome retained the ORF3-E deletion but accumulated mutations in nsp15, nsp16, S, and M genes (FIG. S4D). Engineering the accumulated mutations into ΔORF3-E mNG RNA showed that M mutation T130N conferred multiple rounds of infection on Vero cells (FIG. S4E). Residue T130 is predicted to be on the intra-virion side of the M protein (16, 17), and is conserved in SARS-CoV and SARS-CoV-2 (FIG. S4F). The results indicate that, despite an absence of WT SARS-CoV-2 production, the trans-complementation system could produce mutant virions capable of infecting parental Vero cells for multiple rounds at a barely detectable level, regardless of lacking the entire ORF3 and E genes.

Next, we continuously cultured the S-IV-P5 variant on parental Vero E6 cells for 10 rounds β-4 days per round) to select for potential virions with improved replication efficiency. However, passage did not improve viral replication on Vero cells (FIG. S5). The result suggests that, due to the lack of ORF3 and E genes, the S-IV-P5 virion is unlikely to gain efficient multiple-round amplification on normal cells through adaptation.

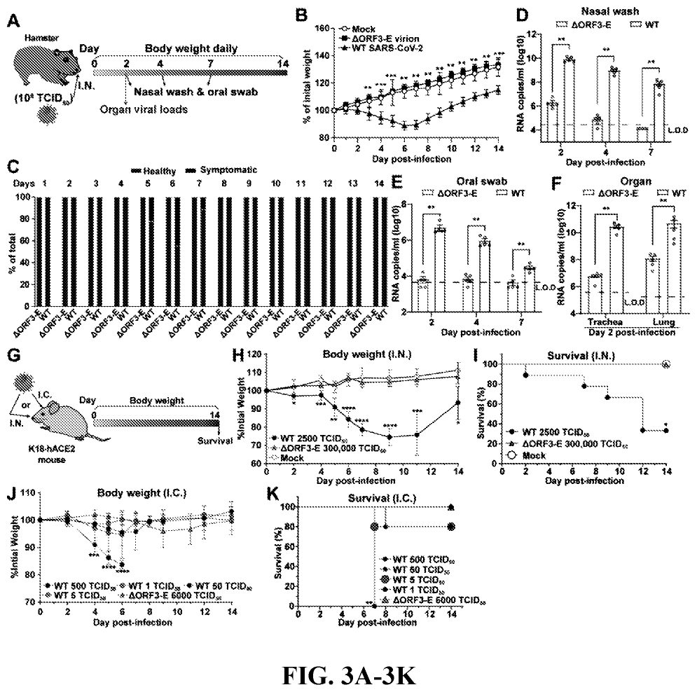

Safety evaluation of ΔORF3-E virions in vivo. We examined the virulence of ΔORF3-E mNG virion in hamsters and K18-hACE2 transgenic mice (18, 19). After intranasal inoculation with 10⁵ TCID₅₀ of ΔORF3-E mNG virion (the highest possible infecting dose; FIG. 3A), hamsters did not lose weight (FIG. 3B) or develop disease (FIG. 3C). In contrast, WT SARS-CoV-2-infected hamsters developed weight loss and mild disease (e.g., ruffled fur). The ΔORF3-E mNG virion-infected hamsters produced low levels of viral RNA in nasal washes (FIG. 3D) and oral swabs (FIG. 3E). Viral RNA levels in the trachea and lungs from the ΔORF3-E virion-infected animals were 50,000- and 400-fold lower than those from the WT virus-infected hamsters, respectively (FIG. 3F). Next, we examined the S-IV-P5 virion, capable of infecting Vero cells for multiple rounds, in hamsters. To maximize the titer of S-IV-P5 virion for infecting animals, we amplified S-IV-P5 on Vero-ORF3-E cells, producing a S-IV-P5 virion stock of 2×10⁵ TCID₅₀/ml. After intranasal inoculation with 5×10³ TCID 50 of S-IV-P5 virion (the highest possible dose), hamsters did not lose weight or develop disease (FIG. S6). Collectively, the results indicate that both ΔORF3-E mNG virion and S-IV-P5 virion are highly attenuated and do not disseminate or cause disease in hamsters.

To corroborate the hamster results, we tested ΔORF3-E mNG virion in more vulnerable K18-hACE2 mice (FIG. 3G). After intranasal inoculation with 3×10⁵ TCID 50 of ΔORF3-E mNG virion (the highest possible dose), K18-hACE2 mice did not lose weight (FIG. 311) or die (FIG. 31); whereas infection with 2.5×10³ TCID₅₀ of WT SARS-CoV-2 resulted in 25% weight loss and 67% lethality. To increase the stringency of the test, we inoculated K18-hACE2 mice by intracranial injection with 6×10³ TCID 50 of ΔORF3-E mNG virion (the highest possible infecting dose); no morbidity (FIG. 3J) or mortality (FIG. 3K) was observed. In contrast, mice inoculated by intracranial route with 500, 50, 5, and 1 TCID₅₀ of WT SARS-CoV-2 developed 100%, 25%, 25%, and 0% mortality, respectively (FIG. 3K). Similar to the ΔORF3-E mNG virion, no morbidity or mortality was observed after mice were inoculated by intranasal or intracranial route with 2.5×10³ or 5×10² TCID₅₀ of S-IV-P5 virion, respectively (FIG. S7). Together, the results demonstrate that both single-round ΔORF3-E mNG virion and multiple-round S-IV-P5 virion lack virulence in K18-hACE2 mice.

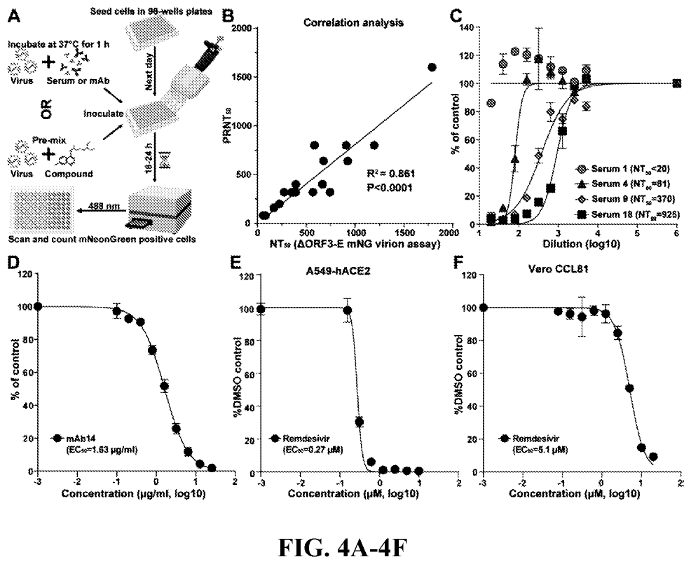

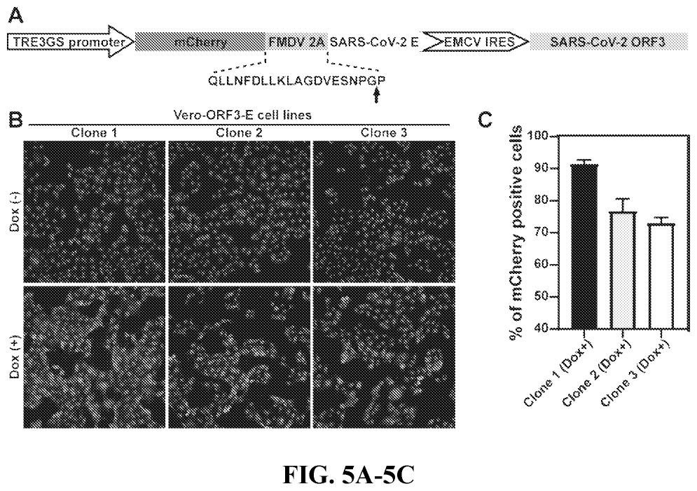

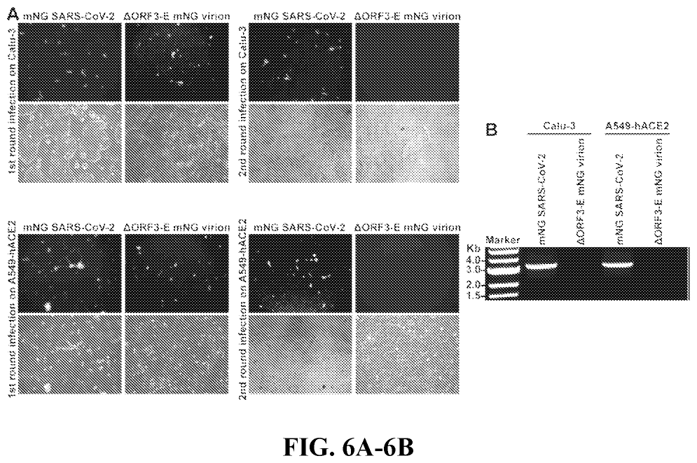

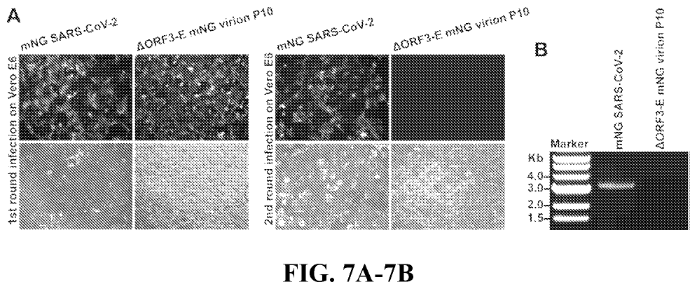

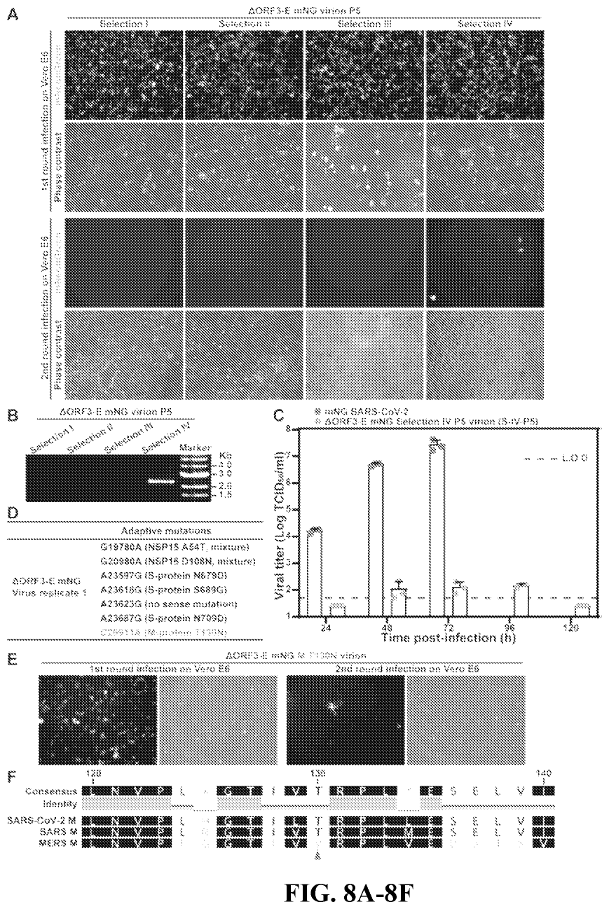

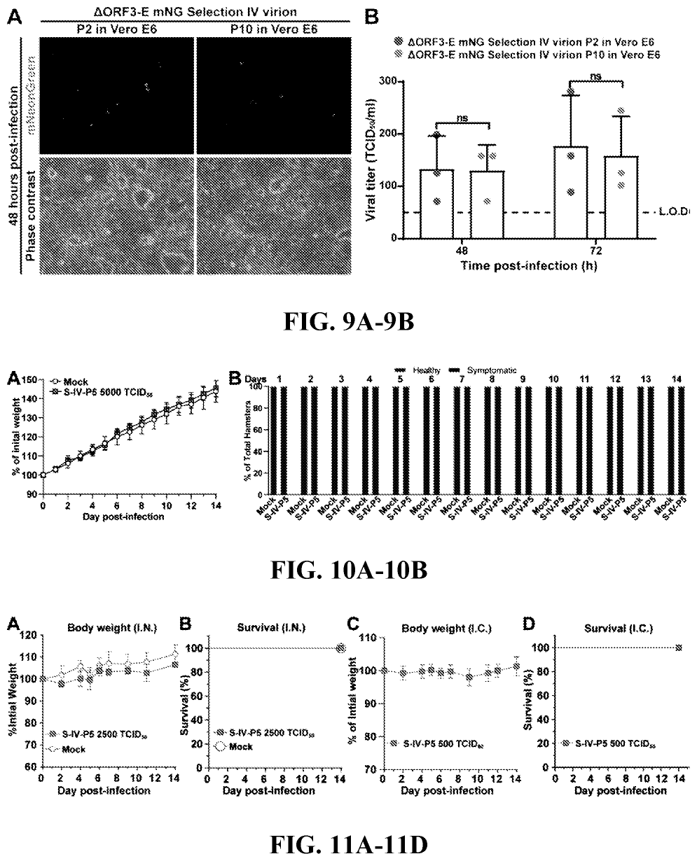

High-throughput neutralization and antiviral testing. We adapted ΔORF3-E mNG virion for a high-throughput neutralization and antiviral assay. FIG. 4A outlines the assay scheme in a 96-well plate format. Neutralization titers of 18 convalescent sera from COVID-19 patients were measured by two assays for comparison: (i) the ΔORF3-E mNG virion assay and (ii) the gold standard plaque reduction neutralization test (PRNT). The two assays produced comparable 50% neutralization titers (NT50) for all specimens (Table 1 and FIG. 4B-C). In addition, the ΔORF3-E mNG virion assay also could be used to measure the 50% effective concentration (EC₅₀) for a monoclonal antibody against SARS-CoV-2 receptor-binding domain (RBD; FIG. 4D). Finally, using remdesivir as an RNA polymerase inhibitor, we evaluated the ΔORF3-E mNG virion assay for antiviral testing. Remdesivir exhibited more potent EC₅₀ on hACE2-A549 cells (0.27 μM; FIG. 4E) than that on Vero cells (5.1 μM; FIG. 4F). The EC₅₀ discrepancy between the two cell types is likely due to different efficiencies in converting remdesivir to its triphosphate form, as previously reported (3, 20). Collectively, the results demonstrate that the ΔORF3-E virion assay can be used for high-throughput neutralization testing and antiviral drug discovery.

TABLE 1
Comparison of neutralization titers between
ΔORF3-E mNG virion and PRNT assays

| | ΔORF3-E | |
|---|---|---|
| Serum ID | virion-NT₅₀ | PRNT₅₀ |
| 1 | <20 | <20 |
| 2 | <20 | <20 |
| 3 | 59.4 | 80 |
| 4 | 81 | 80 |
| 5 | 169 | 160 |
| 6 | 225 | 200 |
| 7 | 274 | 320 |
| 8 | 353 | 320 |
| 9 | 370 | 320 |
| 10 | 392 | 320 |
| 11 | 394 | 400 |
| 12 | 568 | 320 |
| 13 | 585 | 800 |
| 14 | 666 | 400 |
| 15 | 677 | 640 |
| 16 | 744 | 320 |
| 17 | 909 | 800 |
| 18 | 925 | 640 |
| 19 | 1196 | 800 |
| 20 | 1789 | 1600 |

TABLE 2
Primers for plasmids construction and RT-PCR

| Primer name | Sequences (5′ to 3′) | SEQ ID NO |
|---|---|---|
| pcov-F56-F1 | TATACGAAGTTATATTCGATGCGGCCGCGT | 6 |
| | CTCAGAGTGCTTTGGTTTATGATAATAAG | |
| pncov-R5 | TCGCACTAGAATAAACTCTGAACTC | 7 |
| pncov-F6 | AGTTCAGAGTTTATTCTAGTGCGAATAATTG | 8 |
| | CACTTTTGAATATG | |
| pncov-R6 | ATGGCTAGTGTAACTAGCAAGAATACCAC | 9 |
| pncov-F7 | GTATTCTTGCTAGTTACACTAGCCATCCTTA | 10 |
| | CTGCGCTTCG | |
| pncov-R8 | AGGTCGACTCTAGAGGATCC | 11 |
| cov-21115-F | CATTTGTGGGTTTATACAACAAAAG | 12 |
| TRS2-S-R | GAAAAACAAACATTATCCGGTTAGTTGTTAA | 13 |
| | CAAG | |
| TRS2-S-F | CTTGTTAACAACTAACCGGATAATGTTTGTT | 14 |
| | TTTC | |
| S-TRS2-M-R | GAAAAACTAATATAATATTTAATCCGGTTAT | 15 |
| | GTGTAATGTAATTTGACTCCTTTGAGC | |
| TRS2-M-F | CCGGATTAAATATTATATTAGTTTTTCTG | 16 |
| M-TRS2-R | GTAATAAGAAAGCGTCCGGGATGTAGCAAC | 17 |
| | AGTG | |
| M-TRS2-F | CACTGTTGCTACATCCCGGACGCTTTCTTAT | 18 |
| | TAC | |
| ORF6-TRS2-mNG-R | CTTTGCTCACCATATCCGGTTAATCAATCTC | 19 |
| | C | |
| ORF6-TRS2-mNG-F | GGAGATTGATTAACCGGATATGGTGAGCAA | 20 |
| | AG | |
| ORF7-TRS2-ORF8-R | CAAGAAATTTCATATCCGGTTAGGCGTGAC | 21 |
| | AAG | |
| ORF7-TRS2-ORF8-F | CTTGTCACGCCTAACCGGATATGAAATTTCT | 22 |
| | TG | |
| ORF8-TRS2-N-R | CATTATCAGACATTTTAGTTTATCCGGTTAG | 23 |
| | ATGAAATCTAAAACAACACGAACGTC | |
| TRS2-N-F | CCGGATAAACTAAAATGTCTGATAATGG | 24 |
| cov-28501-R | GGTGTTAATTGGAACGCCTTGTCC | 25 |
| M-T130N-F | CCATGGCACTATTCTGAACAGACCGCTTCT | 26 |
| | AGAAAG | |
| M-T130N-R | CTTTCTAGAAGCGGTCTGTTCAGAATAGTG | 27 |
| | CCATGG | |
| 5′UTR-TRS2-F | GATCTGTTCTCTAACCGGATTTTAAAATCTG | 28 |
| | TGTG | |
| 5′UTR-TRS2-R | CACACAGATTTTAAAATCCGGTTAGAGAACA | 29 |
| | GATC | |
| EcoR1-mCherry-F | CACTTCCTACCCTCGTAAAGAATTCGCCAC | 30 |
| | CATGGTGAGCAAGGGCGAGGAG | |
| F2A-optE-R | GACACAAAAGAATACATTGGCCCAGGGTTG | 31 |
| | GACTCGAC | |
| F2A-optE-F | CCCTGGGCCAATGTATTCTTTTGTGTCTGAA | 32 |
| | G | |
| EcoR1-Cov-optE-R | GGGGAGGGAGAGGGGGGGGAATTCCTACA | 33 |
| | CCAGCAGGTCGGGGACC | |
| EcoR1-IRES-F | TAGGAATTCCCGCCCCTCTCCCTCCCCCC | 34 |
| EMCV-IRES-R | ATTATCATCGTGTTTTTCAAAGGAAAACCAC | 35 |
| | G | |
| IRES-optORF3-F | GTTTTCCTTTGAAAAACACGATGATAATATG | 36 |
| | GACCTGTTCATGAGAATC | |
| BamH1-Cov-optORF3-R | CTCGCAGGGGAGGTGGTCTGGATCCCTCA | 37 |
| | CAGAGGAACAGATGTGGTGG | |
| CoV-T7-N-F | ACTGTAATACGACTCACTATAGGATGTCTGA | 38 |
| | TAATGGACCCCAAAATC | |
| polyT-N-R | (T)₃₇AGGCCTGAGTTGAGTCAGCAC | 39 |
| CoV19-N2-F | TTACAAACATTGGCCGCAAA | 40 |
| CoV19-N2-R | GCGCGACATTCCGAAGAA | 41 |

### Methods

Cell lines. Vero E6, Vero CCL-81, Calu-3, and HEK-293T cells were purchased from the American Type Culture Collection (ATCC) and cultured in high-glucose Dulbecco's modified Eagle's medium (DMEM) supplemented with 2 mM L-glutamine, 100 U/ml Penicillium-Streptomycin (P/S), and 10% fetal bovine serum (FBS; HyClone Laboratories, South Logan, UT). Vero-ORF3-E cells were maintained in DMEM medium supplemented with 2 mM L-glutamine, 100 U/ml P/S, 10% FBS, 0.075% sodium bicarbonate, and 10 μg/ml puromycin. The A549-hACE2 cells were generously provided by Shinji Makino (32) and grown in the culture medium supplemented with 10 μg/mL blasticidin at 37° C. with 5% CO2. Medium and other supplements were purchased from Thermo Fisher Scientific (Waltham, MA).

Hamsters. The Syrian hamsters (HsdHan:AURA strain) were purchased from Envigo. Heterozygous K18-hACE c57BL/6J mice (strain: 2B6.Cg-Tg(K18-ACE2)2Prlmn/J) were obtained from The Jackson Laboratory (Bar Harbor, Maine). Animals were housed in groups and fed standard chow diets. Hamster experiments were performed as described previously (33). Briefly, 10⁵ TCID₅₀ in 100 μl of mNG SARS-CoV-2, ΔORF3-E mNG virion, or ΔORF3-E mNG Selection IV P5 (S-IV-P5) virion were inoculated into four- to five-week-old male Syrian golden hamsters via the intranasal route. Ten hamsters were used in SARS-CoV-2- and ΔORF3-E mNG virion-infected groups and 5 hamsters were used in ΔORF3-E mNG S-IV-P5 virion-infected group. From day 1 to 14 post-infection, hamsters were observed daily for weight change and signs of illness. Five hamsters in mNG SARS-CoV-2-, ΔORF3-E mNG virions-, or mock-infected group were sacrificed on day 2 post-infection for lung and trachea collections. Nasal washes and oral swabs of the rest 5 hamsters per group were collected on days 2, 4, and 7 post-infection.

Mice. Animal studies were carried out in accordance with the recommendations in the Guide for the Care and Use of Laboratory Animals of the National Institutes of Health. The protocols were approved by the Institutional Animal Care and Use Committee at the Washington University School of Medicine (assurance number A3381-01). Heterozygous K18-hACE c57BL/6J mice (strain: 2B6.Cg-Tg(K18-ACE2)2Prlmn/J) were obtained from the Jackson Laboratory. Animals were randomized upon arrival at Washington University and housed in groups of <5 per cage in rooms maintained between 68-74° F. with 30-60% humidity and day/night cycles of 12 h intervals (on 6 AM-6 PM). Mice were fed standard chow diets. Mice 7-9 weeks of age and of both sexes were used for this study. Intranasal virus inoculations (50 uL/mouse) were performed under sedation with ketamine hydrochloride and xylazine while intracranial virus inoculations (10 μL/mouse) were performed under sedation with isoflurane; all efforts were made to minimize animal suffering.

Plasmid construction. Seven previously reported subclone plasmids for the assembly of the entire genome of SARS-CoV-2 were used in this study, including pUC57-F1, pCC1-F2, pCC1-F3, pUC57-F4, pUC57-F5, pUC57-F6, and pCC1-F7-mNG (2, 25). For the convenience of deleting ORF3-E gene, we constructed F5, F6, and F7 fragments into one plasmid. F5, F6, and F7-mNG fragments were amplified from corresponding subclones via PCR with primer pairs pcov-F56-F1/pncov-R5, pncov-F6/pncov-R6, and pncov-F7/pncov-R8, respectively (Table 2). All PCR products were cloned together into a pCC1 vector through NotI and ClaI restriction sites using the standard restriction digestion-ligation cloning, resulting in subclone pCC1-F567-mNG.

To introduce ORF3-E deletion and mutant Transcription Regulatory Sequence (TRS) into pCC1-F567-mNG, seven fragments were amplified with primer pairs cov-21115-F/TRS2-S-R, TRS2-S-F/S-TRS2-M-R, TRS2-M-F/M-TRS2-R, M-TRS2-F/ORF6-TRS2-mNG-R, ORF6-TRS2-mNG-F/ORF7-TRS2-ORF8-R, ORF7-TRS2-ORF8-F/ORF8-TRS2-N-R, and TRS2-N-F/cov-28501-R. The seven PCR products were assembled into the pCC1-F567-mNG plasmid that were pre-linearized with NheI and XhoI by using the NEBuilder® HiFi DNA Assembly kit (NEB) according to the manufacturer's instruction, resulting in subclone pCC1-F567-mNG-ΔORF3-E. Mutation T130N in M protein was engineered into pCC1-F567-mNG-ΔORF3-E with primers M-T130N-F/M-T130N-R via overlap PCR. Mutant TRS was engineered into pCC1-F1 with primers 5′UTR-TRS2-F and 5′UTR-TRS2-R via overlap PCR.

For making Vero-ORF3-E cell lines, codon-optimized SARS-CoV-2 ORF3 and E genes were synthesized by GenScript Biotech (Piscataway, NJ). An mCherry reporter Zika virus cDNA plasmid (34) was used as a template to amplify the mCherry-F2A gene. For constructing a lentiviral plasmid expressing ORF3 and E protein of SARS-CoV-2, DNA fragments encoding mCherry-F2A, SARS-CoV-2 E, EMCV IRES, and SARS-CoV-2 ORF3 were amplified with primers EcoR1-mCherry-F/F2A-optE-R, F2A-optE-F/EcoR1-Cov-optE-R, EcoR1-IRES-F/EMCV-IRES-R, and IRES-optORF3-F/BamH1-Cov-optORF3-R, respectively. The PCR products then were inserted into a Tet-on inducible lentiviral vector pLVX (Takara, Mountain View, CA) through EcoRI and BamHI restriction sites, resulting in plasmid pLVX-ORF3-E.

Selection of Vero-ORF3-E cell line. For packaging the lentivirus, the pLVX-ORF3-E plasmid was transfected into HEK-293T cells using the Lenti-X Packaging Single Shots kit (Takara). Lentiviral supernatants were harvested at 72 h post-transfection and filtered through a 0.22 μM membrane (Millipore, Burlington, MA). One day before transduction, Vero E6 cells were seeded in a 6-well plate (3×10⁵ per well) with DMEM medium containing 10% FBS. After 12-18 h, cells were transduced with 2 ml lentivirus for 24 h in the presence of 12 μg/ml of polybrene (Sigma-Aldrich, St. Louis, MO). At 24 h post-transduction, cells from a single well were split into four 10 cm dishes and cultured in medium supplemented with 25 μg/ml of puromycin. The culture medium containing puromycin was refreshed every 2 days. After 2-3 weeks of selection, visible puromycin-resistant cell colonies were formed. Several colonies were transferred into 24-well plates. When confluent, cells were treated with trypsin and seeded in 6-well plates for further expansion. The resulting cells were defined as Vero-ORF3-E P0 cells. For cell line verification, total cellular mRNA was isolated and subject to RT-PCR with primers EcoR1-mCherry-F and BamH1-Cov-optORF3-R (Table 2), followed by cDNA sequencing of the ORF3-E genes.

ΔORF3-E mNG cDNA assembly and in vitro RNA transcription. Full-length genome assembly and RNA transcription were performed as described previously with minor modifications (2). Briefly, individual subclones containing fragments of ΔORF3-E mNG viral genome were digested with appropriated restriction endonucleases and resolved in a 0.8% agarose gel. Specifically, the plasmids containing F1, F2, F3, or F4 fragments were digested with BsaI enzyme, and the plasmid containing F567-mNG-ΔORF3-E fragment was digested with EspI enzyme. All fragments were recovered using the QIAquick Gel Extraction Kit (QIAGEN, Hilden, Germany), and total of 5 μg of the five fragments was ligated in an equal molar ratio by T4 DNA ligase (New England Biolabs, Ipswich, MA) at 4° C. overnight. Afterward, the assembled full-length genomic cDNA was purified by phenol-chloroform extraction and isopropanol precipitation. ΔORF3-E mNG RNA transcripts were generated using the T7 mMessage mMachine kit (Ambion, Austin, TX). To synthesize the N gene RNA transcript of SARS-CoV-2, the N gene was PCR amplified by primers CoV-T7-N-F and polyT-N-R (Table 2) from a plasmid containing the F7 fragment (2); the PCR product was then used for in vitro transcription using the T7 mMessage mMachine kit (Ambion).

ΔORF3-E mNG virion production and quantification. Vero-ORF3-E cells were seeded in a T175 flask and grown in DMEM medium with 100 ng/ml of doxycycline. On the next day, 40 μg of ΔORF3-E mNG RNA and 20 μg of N-gene RNA were electroporated into 8×10⁶ Vero-ORF3-E cells using the Gene Pulser XCell electroporation system (Bio-Rad, Hercules, CA) at a setting of 270V and 950 g with a single pulse. The electroporated cells were then seeded in a T75 flask and cultured in the medium supplemented with doxycycline (Sigma-Aldrich) at 37° C. for 3-4 days. Virion infectivity was quantified by measuring the TCID₅₀ using an end-point dilution assay as previously reported (35). Briefly, Vero-ORF3-E cells were plated on 96-well plates (1.5×10⁴ per well) one day prior to infection. The cells were cultured in medium with doxycycline as described above. ΔORF3-E mNG virions were serially diluted in DMEM medium supplemented with 2% FBS, with 6 replicates per concentration. Cells were infected with 100 μl of diluted virions and incubated at 37° C. for 2-3 days. mNG-positive cells were counted under a fluorescence microscope (Nikon, Tokyo, Japan). TCID₅₀ was calculated using the Reed & Muench method (36).

To assess viral RNA levels, a quantitative RT-PCR assay was conducted using an iTaq Universal SYBR Green one-step kit (Bio-Rad) on a QuantStudio 7 Flex Real-Time PCR Systems (Thermo fisher) by following the manufacturers' protocols. Primers CoV19-N2-F and CoV19-N2-R (Table 2) targeting the N gene were used. Absolute RNA copies were determined by standard curve method using in vitro transcribed RNA containing genomic nucleotide positions 26,044 to 29,883 of the SARS-CoV-2 genome.

RNA extraction, RT-PCR, and cDNA sequencing. Supernatants of infected cells were collected and centrifuged at 1,000 g for 10 min to remove cell debris. Clarified culture fluids (250 μl) were mixed thoroughly with 1 ml of TRIzol LS reagent (Thermo Fisher Scientific). Extracellular RNA was extracted per manufacture's instruction and resuspended in 20 μl of nuclease-free water. RT-PCR was performed using the SuperScript® IV One-Step RT-PCR kit (Thermo Fisher Scientific). Nine cDNA fragments (gF1 to gF9) covering the whole viral genome were generated with specific primers according to the protocol described previously (2). Afterward, cDNA fragments were separated in a 0.8% agarose gel, purified using QIAquick Gel Extraction Kit (QIAGEN), and subjected to Sanger sequencing.

ΔORF3-E mNG virion neutralization assay. Vero CCL-81 cells (1.2×10⁴) in 50 μl of DMEM containing 2% FBS and 100 U/ml P/S were seeded in each well of black μCLEAR flat-bottom 96-well plate (Greiner Bio-one™, Kremsmiinster, Austria). At 16 h post-seeding, 30 μL of 2-fold serial diluted human sera were mixed with 30 μL of ΔORF3-E mNG virion (MOI of 5) and incubated at 37° C. for 1 h. Afterward, 50 μL of virus—sera complexes were transferred to each well of the 96-well plate. After incubating the infected cells at 37° C. for 20 h, 25 μl of Hoechst 33342 Solution (400-fold diluted in Hank's Balanced Salt Solution; Thermo Fisher Scientific) were added to each well to stain the cell nucleus. The plate was sealed with Breath-Easy sealing membrane (Diversified Biotech, Dedham, MA), incubated at 37° C. for 20 min, and quantified for mNG-positive cells using the CellInsight CX5 High-Content Screening Platform (Thermo Fisher Scientific). Infection rates were determined by dividing the mNG-positive cell number to the total cell number. Relative infection rates were obtained by normalizing the infection rates of serum-treated groups to those of non-serum treated controls. The curves of the relative infection rates versus the serum dilutions (log 10 values) were plotted using Prism 9 (GraphPad, San Diego, CA). A nonlinear regression method was used to determine the dilution fold that neutralized 50% of mNG fluorescence (NT50). Each serum was tested in duplicates.

ΔORF3-E mNG virion for mAb and antiviral testing. Vero CCL-81 cells (1.2×10⁴) or A549-hACE2 cells in 50 μl of culture medium containing 2% FBS were seeded in each well of black μCLEAR flat-bottom 96-well plate. At 16 h post-seeding, 2- or 3-fold serial diluted human mAb14 (37) or Remdesivir were mixed with ΔORF3-E mNG virion (MOI of 1). Fifty microliters of mixtures were transferred to each well of the 96-well plate. After incubating the infected cells at 37° C. for 20 h, 25 μl of Hoechst 33342 Solution (400-fold diluted in Hank's Balanced Salt Solution) were added to each well to stain the cell nucleus. The plate was sealed with Breath-Easy sealing membrane, incubated at 37° C. for 20 min. mNG-positive cells were quantified and infection rates were calculated as described above. Relative infection rates were obtained by normalizing the infection rates of treated groups to those of non-treated controls. For Remdesivir, 0.1% of DMSO-treated groups were used as controls. A nonlinear regression method was used to determine the concentration that inhibited 50% of mNG fluorescence (EC₅₀). Experiments were performed in triplicates or quadruplicates.

Biosafety. All aspects of this study were approved by the Office of Environmental Health and Safety at the University of Texas Medical Branch at Galveston before the initiation of this study. Experiments with SARS-CoV-2, trans-complementation, and ΔORF3-E mNG virion were performed in a BSL-3 laboratory by personnel equipped with powered air-purifying respirators.

Transmission Electron Microscopy. Supernatants of infected cells were centrifuged for 10 min at 3,000 g to remove cellular debris. Nickel grids were incubated with clarified supernatants for 10 min followed by glutaraldehyde fixation and 2% uranyl acetate staining. Micrographs were taken using a JEM 14000 (JEOL USA Inc.). Multiple randomly selected fields were imaged.

Bioinformatics analysis. Fluorescence images were processed using ImageJ (38). Virus sequences were download from the NCBI database and aligned using Geneious software. DNA gel images were analyzed using Image Lab software. Statistical graphs or charts were created using the GraphPad Prism 9 software. Figures were created and assembled using BioRender and Adobe illustration (San Jose, CA).

Statistical analysis. A linear regression model in the software Prism 9 (GraphPad) was used to calculate the NT50 and EC₅₀ values from the ΔORF3-E virion assay. Pearson correlation coefficient and two-tailed p-value are calculated using the default settings in the software Prism 9. An unpaired T-test (for two-groups comparison) and ANOVA test (for multi-group comparison) were used in statistical analysis (*, P<0.05, significant; **, P<0.01, very significant; ***, P<0.001, highly significant; ns, P>0.05, not significant).

### REFERENCES

1. B. Hu, H. Guo, P. Zhou, Z. L. Shi, Characteristics of SARS-CoV-2 and COVID-19. Nat Rev Microbiol, (2020).
2. X. Xie et al., An Infectious cDNA Clone of SARS-CoV-2. Cell Host Microbe 27, 841-848 e843 (2020).
3. X. Xie et al., A nanoluciferase SARS-CoV-2 for rapid neutralization testing and screening of anti-infective drugs for COVID-19. Nat Commun 11, 5214 (2020).
4. M. J. Mulligan et al., Phase I/II study of COVID-19 RNA vaccine BNT162b1 in adults. Nature 586, 589-593 (2020).
5. Y. J. Hou et al., SARS-CoV-2 Reverse Genetics Reveals a Variable Infection Gradient in the Respiratory Tract. Cell 182, 1-18 (2020).
6. T. Thi Nhu Thao et al., Rapid reconstruction of SARS-CoV-2 using a synthetic genomics platform. Nature 582, 561-565 (2020).
7. H. Yao et al., Molecular Architecture of the SARS-CoV-2 Virus. Cell 183, 730-738 e713 (2020).
8. F. Ge, Y. Luo, P. X. Liew, E. Hung, Derivation of a novel SARS-coronavirus replicon cell line and its application for anti-SARS drug screening. Virology 360, 150-158 (2007).
9. A. A. Khromykh, E. G. Westaway, Subgenomic replicons of the flavivirus Kunjin: construction and applications. J Virol. 71, 1497-1505 (1997).
10. V. Lohmann et al., Replication of subgenomic hepatitis C virus RNAs in a hepatoma cell line. Science 285, 110-113 (1999).
11. L. Lo, M. Tilgner, P.-Y. Shi, A potential high-throughput assay for screening inhibitors of West Nile virus replication. J. Virol. 77, 12901-12906 (2003).
12. T. Hertzig et al., Rapid identification of coronavirus replicase inhibitors using a selectable replicon RNA. J Gen Virol 85, 1717-1725 (2004).
13. H. Xia et al., Evasion of Type I Interferon by SARS-CoV-2. Cell Rep 33, 108234 (2020).
14. R. L. Graham, D. J. Deming, M. E. Deming, B. L. Yount, R. S. Baric, Evaluation of a recombination-resistant coronavirus as a broadly applicable, rapidly implementable vaccine platform. Commun Biol 1, 179 (2018).
15. B. Yount, R. S. Roberts, L. Lindesmith, R. S. Baric, Rewiring the severe acute respiratory syndrome coronavirus (SARS-CoV) transcription circuit: engineering a recombination-resistant genome. Proc Natl Acad Sci USA 103, 12546-12551 (2006).
16. R. Mahtarin et al., Structure and dynamics of membrane protein in SARS-CoV-2. J Biomol Struct Dyn, 1-14 (2020).
17. S. Thomas, The Structure of the Membrane Protein of SARS-CoV-2 Resembles the Sugar Transporter SemiSWEET. Pathog Immun 5, 342-363 (2020).
18. E. S. Winkler et al., SARS-CoV-2 infection of human ACE2-transgenic mice causes severe lung inflammation and impaired function. Nat Immunol 21, 1327-1335 (2020).
19. J. F. Chan et al., Simulation of the clinical and pathological manifestations of Coronavirus Disease 2019 (COVID-19) in golden Syrian hamster model: implications for disease pathogenesis and transmissibility. Clin Infect Dis, (2020).
20. A. J. Pruijssers et al., Remdesivir Inhibits SARS-CoV-2 in Human Lung Cells and Chimeric SARS-CoV Expressing the SARS-CoV-2 RNA Polymerase in Mice. Cell Rep 32, 107940 (2020).
21. E. E. Walsh et al., Safety and Immunogenicity of Two RNA-Based Covid-19 Vaccine Candidates. N Engl J Med, (2020).
22. A. T. Widge et al., Durability of Responses after SARS-CoV-2 mRNA-1273 Vaccination. N Engl J Med, (2020).
23. J. B. Case et al., Neutralizing Antibody and Soluble ACE2 Inhibition of a Replication-Competent VSV-SARS-CoV-2 and a Clinical Isolate of SARS-CoV-2. Cell Host Microbe 28, 475-485 e475 (2020).
24. C. Zeng et al., Neutralizing antibody against SARS-CoV-2 spike in COVID-19 patients, health care workers, and convalescent plasma donors. JCI Insight 5, (2020).
25. A. E. Muruato et al., A high-throughput neutralizing antibody assay for COVID-19 diagnosis and vaccine evaluation. Nat Commun 11, 4059 (2020).
26. K. Kupferschmidt, Fast-spreading U.K. virus variant raises alarms. Science 371, 9-10 (2021).
27. Xie, X. et al., Neutralization of N501Y mutant SARS-CoV-2 by BNT162b2 vaccine-elicited sera. BioRxiv, doi: URL doi.org/10.1101/2021.1101.1107.425740 (2021).
28. C. Castano-Rodriguez et al., Role of Severe Acute Respiratory Syndrome Coronavirus Viroporins E, 3a, and 8a in Replication and Pathogenesis. mBio 9, (2018).
29. S. Y. Lau et al., Attenuated SARS-CoV-2 variants with deletions at the S1/S2 junction. Emerg Microbes Infect 9, 837-842 (2020).
30. W. B. Klimstra et al., SARS-CoV-2 growth, furin-cleavage-site adaptation and neutralization using serum from acutely infected hospitalized COVID-19 patients. J Gen Virol 101, 1156-1169 (2020).
31. B. A. Johnson et al., Furin Cleavage Site Is Key to SARS-CoV-2 Pathogenesis. bioRxiv, (2020).
32. E. C. Mossel et al., Exogenous ACE2 expression allows refractory cell lines to support severe acute respiratory syndrome coronavirus replication. J Virol 79, 3846-3850 (2005).
33. J. A. Plante et al., Spike mutation D614G alters SARS-CoV-2 fitness. Nature, (2020). 334. C. Shan et al., A live-attenuated Zika virus vaccine candidate induces sterilizing immunity in mouse models. Nat Med, (2017).
35. B. D. Lindenbach, Measuring HCV infectivity produced in cell culture and in vivo. Methods Mol Biol 510, 329-336 (2009).
336. L. J. Reed, H. Muench, A simple method of estimating fifty percent endpoints. The American Journal of Hygiene 27, 493-497 (1938).
37. Z. Ku et al., Molecular determinants and mechanism for antibody cocktail preventing SARS-CoV-2 escape. Nature Communications, URL doi.org/10.1038/s41467-41020-20789-41467 (2021).
38. C. A. Schneider, W. S. Rasband, K. W. Eliceiri, NIH Image to ImageJ:25 years of image analysis. Nat Methods 9, 671-675 (2012).

1. A trans-complementation system comprising:
(i) a ΔORF3/E SARS-CoV-2 genomic viral RNA having ORF3 and envelope genes deleted; and
(ii) a stable producer cell line expressing the SARS-CoV-2 ORF3 and envelope genes, wherein the producer cell line expresses a SARS-CoV-2 ORF3 gene and a SARS-CoV-2 Envelope gene.

2. The trans-complementation system of claim 1, wherein the ΔORF3/Envelope SARS-CoV-2 genomic viral RNA further comprises a heterologous nucleic acid segment encoding a reporter gene.

3. The trans-complementation system of claim 1, wherein the expression of the SARS-CoV-2 ORF3 gene and a SARS-CoV-2 Envelope gene is inducible.

4. A replication defective SARS-CoV-2 RNA genome comprising a deletion of the ORF3 and envelope genes (ΔORF3/E SARS-CoV-2).

5. The replication defective SARS-CoV-2 RNA genome of claim 4, further comprising a mutated transcription regulatory sequence (TRS) comprising a nucleic acid sequence of CCGGAT.

6. The replication defective SARS-CoV-2 RNA genome of claim 4, further comprising a reporter gene.

7. A producer cell comprising at least one heterologous nucleic acid encoding a ORF3 gene and/or a SARS-CoV-2 gene.

8. The producer cell of claim 7, wherein the ORF3 gene and the envelope gene are encoded on the same heterologous nucleic acid.

9. The producer cell of claim 7, wherein the ORF3 gene is encoded on a first heterologous nucleic acid and the envelope gene is encoded on a second heterologous nucleic acid.

10. A method for producing non-replicative SARS-CoV-2 virus comprising, introducing a ΔORF3-E SARS-CoV-2 genomic RNA into ORF3-E SARS-CoV-2 expressing producer cell, wherein the cell produces a non-replicating SARS-CoV-2 virus containing the ΔORF3-E SARS-CoV-2 genomic RNA.

11. A kit comprising:
(i) a replication defective SARS-CoV-2 genome; and
(ii) a producer cell line that complements the replication defective SARS-CoV-2 genome.

12. The kit of claim 11, wherein the replication defective SARS-CoV-2 genome is a ΔORF3/Envelope SARS-CoV-2 genome.

13. An expression cassette comprising:
(i) an inducible promoter operably coupled to ORF3 and E genes;
(ii) an mCherry gene configured to produce a mCherry/E fusion protein upon transcription and translation;
(iii) an RNA segment encoding an auto-cleavage site positioned between the mCherry gene and the E gene; and
(iv) an internal ribosome entry site positioned at the 5′ end of the ORF3 gene.

14. The expression cassette of claim 13, wherein the inducible promoter is a TRE3GS promoter.

15. The expression cassette of claim 13, wherein the auto-cleavage site is a foot-and-mouth disease virus 2A (FMDV 2A) autocleavage site.

16. The expression cassette of claim 13, wherein, the internal ribosome entry site is an encephalomyocarditis virus internal ribosomal entry site (EMCV IRES).

17. The expression cassette of claim 13, further comprised in a viral vector.

18. The expression cassette of claim 17, wherein the viral vector is a lentivirus vector.

19. A stable cell line comprising the expression cassette of claim 13, wherein the expression cassette is stably integrated into the cell line.

20. The stable cell line of claim 19, wherein the cell line is a Vero E6 cell line.


SEQUENCE LISTING (6 sequences)

SEQ ID NO: 1 - DNA, 29903 bp, Artificial Sequence
  Recombinant polynucleotide
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 29392 bp, Artificial Sequence
  Recombinant polynucleotide
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaccggataatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaccggattaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcccggacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaccggatatggcttccaaggtgtacgaccccgagcaacgcaaacgcatgatcactgggcctcagtggtgggctcgctgcaaacaaatgaatgttctggactccttcattaactactatgattccgaaaagcacgccgagaacgccgtgatttttctgcatggtaacgctgccagtagctacctgtggaggcacgtcgtgcctcacatcgaacccgtggctagatgcatcatcccagatctgatcggaatgggaaaatccggcaagagcgggaatggctcatatcgcctcctggatcactacaagtacctcaccgcttggttcgagctgctgaaccttccaaagaaaatcatcttcgtaggccacgactggggggcttgtctggcatttcactactcctacgagcaccaagacaagattaaggccatcgtccatgctgagagtgttgttgacgtgatcgagtcctgggacgagtggcctgacatagaggaggatattgccttaatcaagtctgaggagggtgagaaaatggtgcttgagaataacttcttcgtcgagaccatgctcccatcaaaaattatgcggaaactggaaccagaggagttcgcagcttacctggagccattcaaagagaagggagaggttagacggcctaccctctcttggcctcgcgaaatccctctcgttaagggaggcaagcccgacgtcgtccagattgtccgcaactacaacgcctaccttcgggccagcgacgatctgcctaagatgttcattgaatccgatcctgggttcttttccaacgctattgtcgagggagctaagaagttccctaacactgagttcgtgaaagtgaagggcctccacttcagccaggaggacgctccagatgaaatgggtaagtacatcaagagctttgtggagcgcgttcttaagaacgagcagtgattaattaagaactttcattaattgacttctatttgtgctttttagcctttctgctattccttgttttaattatgcttattatcttttggttctcacttgaactgcaagatcataatgaaacttgtcacgcctaaccggatatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaccggataaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaaa

SEQ ID NO: 3 - DNA, 29173 bp, Artificial Sequence
  Recombinant polynucleotide
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaccggataatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaccggattaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcccggacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaccggatatgtctaagggtgaggaactgttcaccggggtggtgcccatcttagttgagctggatggcgacgtaaacggtcacaagttttctgtgtccggcgaaggagaaggtgatgccacttacggtaagctgacccttaagtttatctgcaccactggcaagttacccgtgccttggcccaccctcgttaccaccctgacctacggcgtgcagtgcttcagccgctaccctgaccacatgaagcagcacgacttcttcaagagtgccatgcccgaaggctacgtccaggagcgtactatcttctttaaggacgatggaaattacaagacccgcgctgaggtgaaattcgaaggcgacaccctggtgaaccgcatcgaactgaaaggcatcgactttaaagaagatggcaacattctggggcacaagttagagtacaactacaacagccacaatgtctatatcatggccgacaaacagaaaaacggcattaaggttaactttaagatccgccataacattgaggatggctctgtgcaactcgccgaccattaccagcagaacacccccatcggtgacggtcctgtgctgcttcccgacaaccactacctgtctacccagtccgctctgagcaaagaccccaacgagaagcgcgatcacatggtcctgctggagttcgtgaccgccgccgggatcactctcggaatggatgaactgtataagtgattaattaagaactttcattaattgacttctatttgtgctttttagcctttctgctattccttgttttaattatgcttattatcttttggttctcacttgaactgcaagatcataatgaaacttgtcacgcctaaccggatatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaccggataaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaaa

SEQ ID NO: 4 - DNA, 29167 bp, Artificial Sequence
  Recombinant polynucleotide
attaaaggtttataccttcccaggtaacaaaccaaccaactttcgatctcttgtagatctgttctctaaccggattttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatctacatgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaccggataatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaccggattaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcccggacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaccggatatggtgagcaaaggagaagaggacaacatggcatcactcccagctacacatgagctgcatatcttcggatccatcaacggagtggacttcgatatggtgggacagggtacagggaacccaaacgacggatacgaggagttgaacctgaagagtaccaagggagacctgcagttctcaccatggatactcgtcccacatataggatacggctttcatcagtacctgccctatccagacggaatgtcacctttccaggcagccatggttgacgggagcggttaccaggtccacaggacaatgcagtttgaggacggagcctcattgaccgtgaactacagatatacctacgaaggaagccatatcaagggagaggctcaagtgaagggaactggattcccagcggacggacccgtgatgaccaacagtctgacggctgcagactggtgcagatccaaaaagacctacccaaatgacaagacaatcataagcaccttcaagtggtcatacactacaggaaacgggaagagatacaggagcactgccagaaccacatacactttcgccaagcctatggctgcaaactacctcaagaaccaacccatgtatgtgttcagaaagacagaactgaagcattctaagaccgaactgaacttcaaggagtggcagaaggcctttactgacgtgatgggaatggacgaactctacaagtaattaattaagaactttcattaattgacttctatttgtgctttttagcctttctgctattccttgttttaattatgcttattatcttttggttctcacttgaactgcaagatcataatgaaacttgtcacgcctaaccggatatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaccggataaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaaa

SEQ ID NO: 5 - DNA, 2794 bp, Artificial Sequence
  Recombinant polynucleotide
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaagaattcgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagcgtacgcagctgttgaattttgaccttctcaagctggcgggagacgtcgagtccaaccctgggccaatgtattcttttgtgtctgaagagacaggcaccctgatcgtgaatagcgtgctgctcttcctggccttcgtggtgttcctgctggtgaccctggctatcctgaccgccctgagactgtgtgcctactgctgcaacatcgtgaacgtgtccctggttaagcctagcttctacgtgtacagcagagtgaagaacctgaacagcagccgggtccccgacctgctggtgtaggaattcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggacctgttcatgagaatcttcaccatcggaaccgtgacactgaaacagggcgagatcaaggacgccacacctagcgacttcgtccgggccaccgccaccatccctatccaggcctctctgccttttggctggctgatcgtgggcgtcgccctgctggccgtgttccagagcgccagcaagattatcaccctgaagaaaagatggcagctggctctgtccaagggcgtgcacttcgtgtgcaacctgctgctgctcttcgtgaccgtgtacagccacctgctgctcgtggccgctggcctggaagcccctttcctgtacctgtacgccctggtgtactttctgcagagcatcaacttcgtgagaatcatcatgcggctgtggctgtgctggaagtgcagaagcaagaaccccctgctttatgatgctaattacttcctgtgttggcacactaactgctacgactactgtatcccctacaatagcgtgacctccagcatcgtgatcacctctggcgacggcaccaccagccctatttctgagcacgattaccagatcggcggctacaccgagaagtgggagagcggagttaaggattgcgtggtgctgcacagctactttacctccgactactaccaactgtattctacacagctgagcacagacaccggcgtggaacacgtgacattcttcatctacaacaagatcgtggacgagcccgaggaacatgtgcagatccacacaatcgacggcagcagcggagtcgtgaaccctgtgatggaacccatctacgatgagccaacaaccaccacatctgttcctctgtga

SEQ ID NO: 6 - DNA, 368 bp, Artificial Sequence
  Recombinant polynucleotide
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaa